SEQUENCE LISTING (8 sequences)

SEQ ID NO: 1 - DNA, 2778 bp, Pseudomonas aeruginosa
  CDS (1)..(2778)
gtgttcaaggatctcggcatcaaggggcgcgtactgctgctcaccctgctccccaccagcctgctggcgatggtgcttggcggttacttcacctgggtccagctgtccgacatgcgcgcccagttgatcgagcgcgggcaactgatcgccgaacaactggcgccgctggccgccaccgcgctggcgcgaaaggataccgccgtgctcaaccgcatcgccaacgaggcgctggaccaaccggacgtgcgcgcggtgaccttcctcgacgcccgccaggaacgcctcgcccatgccgggccaagcatgctcaccgtcgccccggccggcgacgccagccatttgagcatgtccaccgaactggacaccacgcacttcctgctaccggttcttggccgccaccacagcctgtccggcgccaccgagcctgacgacgagcgcgtactcggctgggtcgaactggaactgtcgcaccacgggactctgctgcgcggatatcgcagcctgttcaccagcctcttgctgatcgccgccggcctcggcgtcaccgccctcctcgccctgcgcatgagccgcgcgatcaacgcgccgctggaactgatcagccagggcgtcgcccagctcaaggaaggccgcatggaaacccgcctgccaccgatgggcagcaacgagctggacgaactggcctctggcatcaaccgcatggcggaaacgctgcagagcgcccaggaggaaatgcagcacaacatcgaccaggccaccgaggacgtacggcagaacctggaaaccatcgagatccagaacatcgagctggacctggcgcgcaaggaggccctggaggcgagcaggatcaagtccgagttcctcgccaacatgagccacgagatccgcaccccgctcaacggcatcctcggtttcaccaacctgctgcagaagagcgagctcagcccgcgccagcaggactacctcacgaccatccagaaatcggcggaaagcctgctggggatcatcaacgagatcctcgatttctcgaagatcgaggccggcaagctggttctggaaaacctccctttcaatctccgcgacctgatccaggacgccctgaccatgctggctccggccgcccacgagaagcaactggaactggtcagcctggtctaccgggataccccgatccaattgcagggcgacccgcagcggctgaagcagatcctcaccaacctggtcggcaacgccatcaagttcacccagggcggcaccgtcgccgtacgcgccatgctcgaggacgaaagcgacgaccgcgcgcagctgcggatcagcgtccaggacaccggtatcggcctctccgaggaagaccagcaagccttgttcaaggccttcagccaggccgacaactcactgtcgcggcaagccggtggcaccggcctgggcctggtgatctccaagcgcctgattgagcagatgggcggcgagatcggcgtcgacagtacgcctggggaaggcgccgagttctggatcagcctgagtctgccgaaaagtcgcgacgacaacgaggagccgggcgcctcctgggccgcgggccaacgcgtggcgctgctcgaaccgcaggaactgacgcgccgctcgctgcaccaccagctcaccgacttcggcctggaagtgagcgaattcgccgacctcgacagcctccaggaaagcctgcgcaacccgccgcccggccagttgccgatcagcctggcggtgctcggcgtctcggccgcgatccatccgccggaagagctgagccagtcgttctgggaattcgaacggctcggctgcaagaccctggtgctctgcccgaccaccgagcaggcgcaataccacgcgaccctgcccgacgaacaggtcgaggccaagcccgcctgcacccgcaagctgcaacgcaagctgcaggagttgcttcaagtccgcccgacgcgcagcgacaagccccacgccatggtttccggacggccgccacggctgctatgcgtcgacgacaacccggccaacctgctgctggtgcagaccctgctcagcgacctcggcgcccaggtcaccgcggtggacagcggctacgcggccctcgaggtagtgcagcgcgagcgcttcgacctggtcttcatggacgtgcagatgcccggcatggacggccgccaggccaccgaggcgatccgccgctgggaggccgagcgggaagtcagcccggtgccggtgatcgcgctcaccgcacatgcgctttccaacgagaagcgcgcattgctgcaggccggcatggacgactacctgaccaagccgatcgacgagcagcaattggcccaggtagtgctgaagtggaccggactgagcctgggccagtcgctggccagcatgagccgtgcgccgcagctcggccagttgagcgtgctcgaccccgaggaagggctgcgcctggccgccggcaaggccgacctcgccgccgacatgctggcgatgctgctggcctcgctggcggcggaccgccaggcgattcgccaggcccgcgacaacgacgaccgcaccgctttgctcgagagggtccaccggctgcatggcgccacccgctactgtggcgtgccgcagttgcgcgcggcctgccagaccagcgaaaccctgctcaagcagaacgatccggcggcggccgcggccctggacgagctggacaaggccatcgaggccctggccgacactgcctcggccaccacccacctgtcctccaccagcctcgactccagcgaactctga

SEQ ID NO: 2 - protein, 925 aa, Pseudomonas aeruginosa
VFKDLGIKGRVLLLTLLPTSLLAMVLGGYFTWVQLSDMRAQLIERGQLIAEQLAPLAATALARKDTAVLNRIANEALDQPDVRAVTFLDARQERLAHAGPSMLTVAPAGDASHLSMSTELDTTHFLLPVLGRHHSLSGATEPDDERVLGWVELELSHHGTLLRGYRSLFTSLLLIAAGLGVTALLALRMSRAINAPLELISQGVAQLKEGRMETRLPPMGSNELDELASGINRMAETLQSAQEEMQHNIDQATEDVRQNLETIEIQNIELDLARKEALEASRIKSEFLANMSHEIRTPLNGILGFTNLLQKSELSPRQQDYLTTIQKSAESLLGIINEILDFSKIEAGKLVLENLPFNLRDLIQDALTMLAPAAHEKQLELVSLVYRDTPIQLQGDPQRLKQILTNLVGNAIKFTQGGTVAVRAMLEDESDDRAQLRISVQDTGIGLSEEDQQALFKAFSQADNSLSRQAGGTGLGLVISKRLIEQMGGEIGVDSTPGEGAEFWISLSLPKSRDDNEEPGASWAAGQRVALLEPQELTRRSLHHQLTDFGLEVSEFADLDSLQESLRNPPPGQLPISLAVLGVSAAIHPPEELSQSFWEFERLGCKTLVLCPTTEQAQYHATLPDEQVEAKPACTRKLQRKLQELLQVRPTRSDKPHAMVSGRPPRLLCVDDNPANLLLVQTLLSDLGAQVTAVDSGYAALEVVQRERFDLVFMDVQMPGMDGRQATEAIRRWEAEREVSPVPVIALTAHALSNEKRALLQAGMDDYLTKPIDEQQLAQVVLKWTGLSLGQSLASMSRAPQLGQLSVLDPEEGLRLAAGKADLAADMLAMLLASLAADRQAIRQARDNDDRTALLERVHRLHGATRYCGVPQLRAACQTSETLLKQNDPAAAAALDELDKAIEALADTASATTHLSSTSLDSSEL

SEQ ID NO: 3 - protein, 5 aa, Artificial Sequence
  Artificial Peptide Linker Sequence
GGGGS

SEQ ID NO: 4 - protein, 22 aa, Artificial Sequence
  Artificial Peptide Linker Sequence
GGGGGGSMFGGAKKRSGGGGGG

SEQ ID NO: 5 - DNA, 24 bp, Artificial Sequence
  Forward Primer
gatggtgcttggcggttacttcac

SEQ ID NO: 6 - DNA, 24 bp, Artificial Sequence
  Reverse Primer
acgtccatgaagaccaggtcgaag

SEQ ID NO: 7 - DNA, 23 bp, Artificial Sequence
  Forward Primer
cgccaacccctcttccccgtctc

SEQ ID NO: 8 - DNA, 23 bp, Artificial Sequence
  Reverse Primer
cggcgacagcgtgcggcgaatag

(19) United States
(12) Patent Application Publication
(10) Pub. No.: US 2008/0075730 A1
(43) Pub. Date: Mar. 27, 2008

(54) Methods and compositions for treating biofilms

(75) Inventors: Douglas G. Storey, Calgary (CA); Michael D. Parkins, Calgary (CA); Howard Ceri, Calgary (CA); James A. Davies, Sherwood Park (CA); Merle E. Olson, Calgary (CA); Lyriam L. R. Marques, Calgary (CA)

Correspondence Address: BUCHANAN, INGERSOLL & ROONEY LLP, P.O. BOX 1404, ALEXANDRIA, VA, 22313-1404, US

(73) Assignee: University Technologies International Inc., Calgary (CA)

(21) Appl. No.: 11/652,866

(22) Filed: Jan. 12, 2007

Related U.S. Application Data
(63) Continuation-in-part of application No. 10/828,557, filed on Apr. 21, 2004, now abandoned.
(60) Provisional application No. 60/465,153, filed on Apr. 23, 2003.

(51) Int. Cl. A61K 31/70; A61K 38/00; A61K 39/395; A61P 43/00

(52) U.S. Cl. 424/164.1; 514/12; 514/44

(57) ABSTRACT

This disclosure relates to methods and compositions to regulate biofilm formation. In particular, the disclosure provides methods and compositions that relate to regulation of biofilm formation by modulating the GacA/GacS regulatory system as well as methods and compositions for inhibiting small colony variant formation and reversion of resistant bacteria to a wild-type phenotype.

### CROSS REFERENCE TO RELATED APPLICATIONS

This application claims priority as a continuation-in-part to U.S. application Ser. No. 10/828,557, filed Apr. 21, 2004, which claims priority under 35 U.S.C. §119 from Provisional Application Ser. No. 60/465,153, filed Apr. 23, 2003, the disclosures of which are incorporated herein by reference.

### TECHNICAL FIELD

This invention relates to methods and compositions to overcome reduced antibiotic susceptibility of biofilms. In particular, the invention relates to regulation of biofilm phenotypic plasticity by modulating the GacA/GacS regulatory system.

### BACKGROUND

Biofilms are an alternate mode of bacterial growth where cells exist within a complex and highly heterogeneous matrix of extracellular polymers adherent to a surface. Pathogenic microbial biofilms display decreased susceptibility to antimicrobial agents and elevated resistance to host immune response, often causing chronic infections. Pseudomonas aeruginosa, a gram negative opportunistic pathogen, forms biofilms within the lungs of cystic fibrosis patients and has become the model organism for the study of biofilm physiology. P. aeruginosa utilizes several global regulatory elements to control expression of its vast array of virulence factors. In P. aeruginosa, the GacA/GacS regulon has been shown to include genes which affect production of pyocyanin, cyanide, lipase, C4 homoserine lactone (HSL) and is essential for virulence in three independent models of infection.

However, studies in other organisms such as fluorescent pseudomonades, have implicated much broader ranging effects of the GacA/GacS regulon. In Pseudomonas chlororaphis O6, which is an aggressive colonizer of plant roots under competitive soil conditions, the GacA/GacS two component regulatory system has been demonstrated to control expression of protease, phytotoxins, and secondary metabolites. P. chlororaphis O6 inhibits growth of several fungal pathogens in vitro. The O6 mutant L21, generated by transposon mutagenesis, lacked production of antifungal phenazines. The O6 gacS gene, encoding a sensor kinase, complemented L21, although the Tn5 insertion site was in gene, ppx encoding exopolyphosphatase. O6 gacS mutants, like L21, lacked in vitro production of phenazines, protease, and HSLs. Confocal laser microscopy, revealed that wild-type O6 but not the gacS mutant produced phenazines on bean roots. The gacS mutant had decreased catalase activity and was less competitive than wild-type in colonization of bean roots in the presence of competing microbes.

### SUMMARY

The disclosure provides a method of inhibiting biofilm formation comprising inhibiting the gacA/gacS regulatory system of an organism.

The disclosure also provide a method of inhibiting the production of small colony variants (SCVs) comprising contacting a bacterial population with an antagonist of the gacA/gacS regulatory system of the bacteria.

The disclosure also provides a composition useful for preventing biofilm formation comprising an antagonist of a gacA/gacS regulatory system in a pharmaceutically acceptable form.

The disclosure further provides a method of treating an antimicrobial resistant biofilm. The method includes contacting resistant bacteria in the biofilm comprising a mutation in gacS with a gacS agonist, wherein the gacS agonist generates a wild-type gacS phenotype in the resistant bacteria.

The disclosure provides a composition comprising a gacS agonist and a pharmaceutically acceptable carrier. In a further aspect, the composition comprises a gacS agonist and an antimicrobial agent.

The disclosure also provides a method of inhibiting biofilm formation, comprising: contacting bacterial population with a gacS and/or gacA antagonist; monitoring the bacterial population for the formation of a small colony variant; contacting the small colony variant with a composition comprising gacS agonist.

The disclosure relates to the role of the GacA/GacS two component global regulatory system in biofilm formation of both the opportunistic pathogens (e.g., Pseudomonas aeruginosa and the fluorescent pseudomonad Pseudomonas chlororaphis O6). The GacA/GacS two component regulatory system is a genetic element necessary for biofilm formation in various bacteria. Biofilm growth curves demonstrated that when the response regulator, gacA, was disrupted in P. aeruginosa strain PA14 a 10 fold reduction in biofilm formation capacity resulted relative to wild type PA14 and a toxA derivative. However, no significant difference in the planktonic growth rate of PA14 gacA was observed. Scanning electron microscopy of biofilms formed by PA14 gacA revealed diffuse clusters of cells which failed to aggregate into microcolonies, implying a deficit in biofilm maturation. Twitching motility assays, and C12 homoserine lactone (HSL) autoinducer bioassays reveal normal zones of twitching motility and C12 homoserine lactone (HSL) production, indicating this is not merely an upstream effect on either the las quorum sensing system or type IV pili biogenesis. Furthermore, antibiotic susceptibility profiling has demonstrated PA14 gacA biofilms have moderately decreased resistance to azythromycin, chloramphenicol, erythromycin, piperacillin, and polymixin B relative to either PA14 wild type or the toxA control. This establishes the GacA/GacS two component regulatory system as an independent regulatory element in P. aeruginosa biofilm formation.

The disclosure further demonstrates that the regulatory gacS gene plays an important role in biofilm formation and structure in Pseudomonas chlororaphis O6 (PcO6) using a gacS knock-out mutant generated in PcO6 by Tn-5 insertion. The ability of wild type and mutant strains to form biofilms was evaluated in vitro using the MBEC device. Biofilm formation by the gacS mutant, as evaluated by colony counts and scanning electron microscopy was greatly reduced in comparison with the wild type strain, but it was restored by complementation with an active gacS construct. Given the fact of the gacS involvement in root colonization, the results suggest a plausible role of biofilm formation in PcO6 biocontrol capability.

The details of one or more embodiments of the invention are set forth in the accompanying drawings and the description below. Other features, objects, and advantages of the invention will be apparent from the description and drawings, and from the claims.

### DESCRIPTION OF DRAWINGS

FIG. 1 shows growth curves of PA14 wild-type, Pa14 toxA⁻, and PA14 gacA⁻ grown in the MBEC™ device.

FIG. 2 shows growth curves of PA 14 wild-type, PA14 toxA⁻, and PA14 gacA⁻ transformed with pGacA grown in the MBEC™ Device.

FIG. 3 shows growth curves of PA14 wild-type, PA14 toxA⁻, and PA14 gacA⁻ transformed with control plasmid pUCSF grown in the MBEC™ Device.

FIG. 4 provides scanning electron micrographs of various P. aeruginosa strains.

FIG. 5 shows growth curves of PA14 wild-type, PA14 toxA⁻ and PA14 gacA⁻ transformed with pMJG1.7 (multi-copy lasR) grown in the MBEC™ Device.

FIG. 6 illustrates motility assay results of various P. aeruginosa strains.

FIG. 7 illustrates the production of C12 homoserine lactone (HSL) by various P. aeruginosa strains.

FIG. 8 illustrates P. chlororaphis O6 biofilm growth on MBEC device: (A) a wild type; (B) a gacS knock-out mutant; and (C) a gacS/+−complemented mutant.

FIG. 9A-F provides scanning electron micrographs of P. chlororaphis O6 strains at different cell densities. A and B represent wild type P. chlororaphis at different magnifications showing dense biofilm formation, organization into a microcolony three-dimensional structure typical of biofilm formation. C and D represent different magnifications of SEMs of the gacS mutant showing sparse cell attachment and failure to generate microcolony formation, but rather clusters of small cell groupings with little organized structure. E and F are different magnifications of SEMs of the gacS mutant complemented with the gacS gene in trans. Formation of true biofilm structure returned to the mutant by restoration of an active gacS gene as seen by the microcolony organization into complex architecture typical of a biofilm. Magnification for pictures: Wild type, A=1.1 K, B=3.5 K; Mutant C=1.5 K; D=3.5 K; Complemented strain: E=1.0 K and F=3.5 K.

FIG. 10A-F provides scanning electron micrographs of P. chlororaphis O6 strains at different cell densities. A and B represent wild type P. chlororaphis at different magnifications showing dense biofilm formation, organization into a microcolony three-dimensional structure typical of biofilm formation. C and D represent different magnifications of SEMs of the gacS mutant showing sparse cell attachment and failure to generate microcolony formation, but rather clusters of small cell groupings with little organized structure. E and F are different magnifications of SEMs of the gacS mutant complemented with the gacS gene in trans. Formation of true biofilm structure returned to the mutant by restoration of an active gacS gene as seen by the microcolony organization into complex architecture typical of a biofilm. Magnification for pictures: Wild type, A=1.1 K, B=3.5 K; Mutant C=1.5 K; D=3.5 K; Complemented strain: E=1.0 K and F=3.5 K.

FIG. 11A-J shows characteristics of Pseudomonas aeruginosa PA14 wild-type, gacS⁻ and SCV strains. (a) Growth curves of biofilms on polystyrene pegs in the MBEC assay. (b-d) The swarming motility of the PA14 gacS- strain was much greater than that of the other isogenic strains. (e-g) Similarly, the gacS- strain was significantly more motile than the wild-type strain on swim agar (*P<0.01 by a two-sample t-test, four replicates each). Conversely, PA14 SCV was significantly less motile than the wild-type strain (**P<0.02 by a two-sample t-test, four replicates each). (h-j) Streak plates on LB agar.

FIG. 12A-F shows biofilm formation by Pseudomonas aeruginosa PA14 wild-type, gacS⁻ and SCV strains at 10 h. Biofilms were grown in LB broth (at 35° C. and 150 r.p.m.) in the MBEC Physiology and Genetics (P&G) assay, stained with acridine orange, and then imaged using scanning confocal laser microscopy. (a, c, e) Two-dimensional average projections of images along the z-axis. (b, d, f) Three-dimensional reconstructions of z-stacks pictured on the left and showing surface topology of the biofilms. Each panel represents a square area of 238.1×238.1 mm.

FIG. 13A-F shows biofilm formation by Pseudomonas aeruginosa PA14 wild-type, gacS⁻ and SCV strains at 24 h. Growth conditions and fluorescent staining were identical to those described in the legend of FIG. 12. Images were captured using scanning confocal laser microscopy. (a, c, e) Two-dimensional average projections of images along the z-axis. (b, d, f) Three-dimensional reconstructions of z-stacks pictured on the left and showing surface topology of the biofilms. Each panel represents a square area of 238.1×238.1 mm.

FIG. 14A-D are SEM of biofilms of Pseudomonas aeruginosa PA14 wild-type, gacS⁻ and SCV strains at 27 h. Biofilms were grown in the MBEC high-throughput assay in LB broth (at 35° C. and 3.5 rocks per minute on a rocking table). (a) Biofilm formation by wild-type P. aeruginosa PA14. (b) Biofilm formation by the isogenic P. aeruginosa PA14 gacS mutant. (c and d) Biofilm formation by the SCV strain, showing a break-away section that highlights its hyper-biofilm-forming phenotype in contrast to the other isogenic strains.

FIG. 15A-D shows killing of Pseudomonas aeruginosa PA14 wild-type, gacS⁻ and SCV biofilms by heavy metal cations. Biofilms were exposed to heavy metals for 2 h, rinsed, then disrupted into fresh medium containing neutralizing agents. (a and c) Mean viable cell counts from biofilms exposed to Cu²⁺ and Ag²⁺, respectively. (b and d) Log-killing of biofilms by Cu²⁺ and Ag²⁺, respectively. The SCV produced biofilms that were much more tolerant to heavy metal toxicity than either of the other strains. Solid lines with squares represent the wild-type strain, dashed lines with triangles represent the isogenic gacS⁻ strain, and dotted lines with circles represent the SCV.

FIG. 16A-D shows killing of Pseudomonas aeruginosa PA14 wild-type, gacS⁻ and SCV biofilms by hydrogen peroxide (H₂O₂) and ciprofloxacin. Test conditions were similar to those described in the legend of FIG. 15. (a and c) Mean viable cell counts from biofilms exposed to H2O2 and ciprofloxacin, respectively. (b and d) Log-killing of biofilms by H₂O₂ and ciprofloxacin, respectively. The SCV produced biofilms that were recalcitrant to killing by peroxide or the DNA gyrase inhibitor ciprofloxacin relative to the other strains. Solid lines with squares represent the wild-type strain, dashed lines with triangles represent the isogenic gacS⁻ strain, and dotted lines with circles represent the SCV.

FIG. 17 depicts the gacS/gacA regulatory pathway in biofilm formation and hyper-resistant biofilms.

### DETAILED DESCRIPTION

As used herein and in the appended claims, the singular forms “a,” “and,” and “the” include plural referents unless the context clearly dictates otherwise. Thus, for example, reference to “a bacteria” includes a plurality of such bacteria and reference to “the agent” includes reference to one or more agents known to those skilled in the art, and so forth.

Unless defined otherwise, all technical and scientific terms used herein have the same meaning as commonly understood to one of ordinary skill in the art to which this disclosure belongs. Although methods and materials similar or equivalent to those described herein can be used in the practice of the disclosed methods and compositions, the exemplary methods, devices and materials are described herein.

The publications discussed above and throughout the text are provided solely for their disclosure prior to the filing date of the present application. Nothing herein is to be construed as an admission that the inventors are not entitled to antedate such disclosure by virtue of prior disclosure.

Phase variation is a process of reversible, high-frequency phenotypic switching that is mediated by mutation, reorganization, or modification of DNA. This process is used by several bacterial species to generate population diversity that increases bacterial fitness and is important in niche adaptation. Phase variation can sometimes be observed by the appearance of morphologically distinct colonies or sectors within a colony. In contrast to spontaneous mutations, which occur at a frequency of approximately 10⁻⁷ mutations per cell per generation, phase variation occurs at frequencies higher than 10⁻⁵ switches per cell per generation. Four mechanisms of phase variation are known: (i) slipped-strand mispairing, dependent on variations in the length of a repeat tract, switching a gene on or off as a result of frame shifts, or regulating the level of expression by altering promoter spacing; (ii) genomic rearrangements, based on invertible elements or recombination events resulting in gene conversions; (iii) differential methylation, based on the presence of methylation sites within a promoter, which can regulate the binding of regulatory proteins; and (iv) random unprogrammed variation, which can switch traits on and off via random reversible mutations.

Phase variation has been reported to regulate the production of pili, outer membrane proteins, flagella, fimbriae, surface lipoproteins and other surface-exposed structures, secondary metabolites and secreted enzymes such as proteases, lipases, and chitinases. For example, out of 46 Pseudomonas strains antagonistic against the wheat-pathogenic fungus Geaumannomyces graminis pv. tritici R3-11A, 43 displayed colony phase variation. Estimation of the phase variation frequencies showed approximately 5.0×10⁻⁵ and 9.0×10⁻² switches per cell per generation for phase I to II and for phase II to I, respectively.

GacS shares a high degree of identity with an open reading frame (ORF3) downstream and adjacent to pvrR (phenotype variant regulator), a hypothetical response regulator for a two-component system (PubMed accession number AF482691; incorporated herein by reference). Together, ORF3 and pvrR form a hybrid, putative sensor kinase and response regulator. Overexpression of PvrR from a plasmid reduces the frequency of phenotypic variation in P. aeruginosa biofilms. GacS/GacA are also upstream regulators of the pel (pellicle) operon. This cluster of seven adjacent genes is postulated to encode polysaccharide biosynthetic enzymes important for matrix formation in P. aeruginosa PA14. These genes are implicated in surface adherence, and in general the pel locus shows increased expression in SCVs derived from biofilms of P. aeruginosa PAO1. The data demonstrate that a functional gacS limited the generation of SCVs in biofilms, and that this phenomenon was specific to the gacS mutant, as phenotypically stable SCVs were not produced from an isogenic gacA strain of P. aeruginosa PA14. Further indicative of the low-fidelity relationship between GacA and GacS are decreases in AHSL levels of gacS relative to the gacA strain and the differences in Biofilm structure. Two other sensor kinases, RetS and LadS, are known to modify intracellular signalling through GacA. It is interesting to note that deletion of retS is similarly associated with the occurrence of hyper-biofilm-forming colony morphology variants in P. aeruginosa.

The sensor kinase GacS and the response regulator GacA are members of a two-component system that is present in a wide variety of Gram-negative bacteria and has been studied mainly in enteric bacteria and fluorescent pseudomonads. The GacS/GacA system controls the production of secondary metabolites and extracellular enzymes involved in pathogenicity to plants and animals, biocontrol of soil-borne plant diseases, ecological fitness, or tolerance to stress. A current model proposes that GacS senses a still-unknown signal and activates, via a phosphorelay mechanism, the GacA transcription regulator, which in turn triggers the expression of target genes. The GacS protein belongs to the unorthodox sensor kinases, characterized by an autophosphorylation, a receiver, and an output domain. The periplasmic loop domain of GacS is poorly conserved in diverse bacteria. Thus, a common signal interacting with this domain would be unexpected. Based on a comparison with the transcriptional regulator NarL, a secondary structure can be predicted for the GacA sensor kinases. Certain genes whose expression is regulated by the GacS/GacA system are regulated in parallel by the small RNA binding protein RsmA (CsrA) at a post-transcriptional level. It is suggested that the GacS/GacA system operates a switch between primary and secondary metabolism, with a major involvement of posttranscriptional control mechanisms.

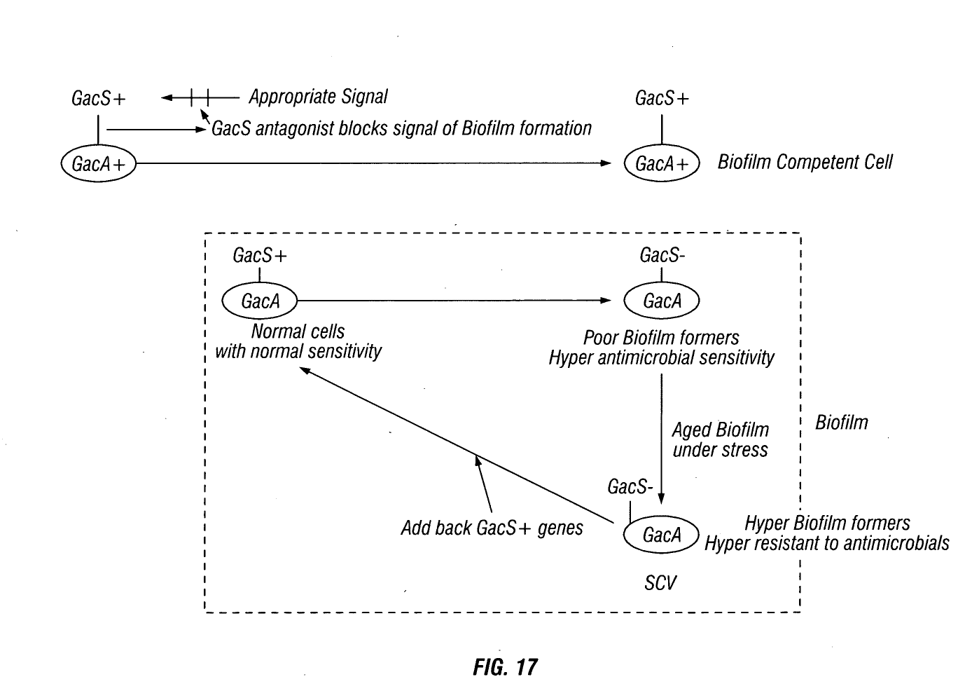

The GacS/GacA two-component regulatory system in pseudomonads regulates genes involved in virulence, secondary metabolism and biofilm formation. Despite these regulatory functions, some Pseudomonas species are prone to spontaneous inactivating mutations in gacA and gacS. A gacS⁻ strain of Pseudomonas aeruginosa PA14 was constructed to study the physiological role of this sensor histidine kinase. This loss-of-function mutation was associated with hypermotility, reduced production of acylhomoserine lactones, impaired biofilm maturation, and decreased antimicrobial resistance. Biofilms of the gacS mutant gave rise to phenotypically stable small colony variants (SCVs) with increasing frequency when exposed to silver cations, hydrogen peroxide, human serum, or certain antibiotics (tobramicin, amikacin, azetronam, ceftrioxone, oxacilin, piperacillin or rifampicin). When cultured, the SCV produced thicker biofilms with greater cell density and greater antimicrobial resistance (“hyper-resistant biofilms”) than did the wild-type or parental gacS strains. Similar to other colony component signal transduction, quorum morphology variants described in the literature, this SCV was less motile than the sensing, small colony variant, antimicrobial wild-type strain and autoaggregated in broth culture. Complementation with gacS in trans restored the ability of the SCV to revert to a normal colony morphotype and lose their hyper-resistance to antimicrobials. These findings indicate that mutation of gacS is associated with the occurrence of stress resistant SCV cells in P. aeruginosa biofilms and suggests that in some instances GacS may be necessary for reversion of these variants to a wild-type state and to wild-type sensitivity to antibiotics and other stressors. FIG. 17 depicts an exemplary process by which the gacS/gacA regulatory system modifies biofilm formation and resistance. For example, FIG. 17 demonstrates that the two components system in bacteria promotes biofilm formation and that stress factors generate gacS mutants the form small colony variants with increased antimicrobial resistance.

Mutation of gacS is not associated with a loss of fitness of pseudomonads in the rhizosphere. Using P. chlororaphis as an example, studies have suggested that mixtures of gacS mutants with the wild-type population may enhance the survival of this bacterium in soil. Preliminary evidence suggests that this may be linked to phenotypic variation. Inactivation of gacS in P. chlororaphis gives rise to highly adherent small colony variants (SCVs) from aged biofilms exposed to silver cations. These isolates are less motile and superior at forming biofilms, which may be an important process for root colonization. GacS/GacA signaling in this microorganism has been implicated in attenuating virulence and establishing chronic infections in the cystic fibrosis (CF) lung. Further, the isolation of colony morphology variants with an increased ability for forming biofilms has been described for many laboratory and clinical strains of P. aeruginosa.

The disclosure demonstrates that in a gacS⁻ environment pseudomonads throw off small colony variants (SCVs) that are better biofilm formers, more resistant to antibiotics and other environmental stresses and allow the biofilm to survive treatments that the wild type pseudomonads can't survive as planktonic bacteria and that these variants are present at much higher rates in biofilms. It should also be pointed out that the wild type pseudomonads can also throw off SCVs but these are not stable and revert back to wild type very quickly. So the difference between wild type and gacS⁻ populations is the stability of the SCVs that possess the antibiotic resistance capabilities. Therefore by reverting the gacS⁻ population spontaneously developed in the biofilm to provide antibiotic resistance to the biofilm to the gacS wild type phenotype the biofilm can be rendered susceptible to antibiotics. Delivery of gacS polynucleotides to biofilms can be performed, for example, in burn and wound patients where polynucleotides are easily delivered but methods include aerosolizing the polynucleotides and the development of carrier systems.

The disclosure demonstrates that phenotypically stable SCVs from aged biofilms of multihost virulent Pseudomonas aeruginosa PA14 bear an inactivating mutation in the sensor kinase gacS. These colony morphology variants were hyper-adherent, less motile, and had a hyperbiofilm-forming phenotype (“hyper-resistant biofilms”) compared with the wild-type strain. These variants also had elevated resistance to antimicrobials. Biofilms of PA14 gacS⁻ gave rise to the SCV phenotype at a higher frequency (than growth controls) when exposed to some clinically used antibiotics, silver ions, or hydrogen peroxide. Furthermore, the phenotypic stability of the SCV strain demonstrates that GacS controls reversion of these colony morphology variants to a wild-type state.

The disclosure contemplates a two step process of biofilm formation and thus a continuum for treatment. Each step can be modulated independently to inhibit biofilm formation and antimicrobial susceptibility (see, e.g., FIG. 17). Thus, in one aspect, the disclosure inhibits biofilm formation (as set forth in FIG. 17) by utilizing gacS/gacA antagonist that reduce biofilm formation through normal microbial metabolism and growth. In another aspect, the disclosure treats biofilm formation by increasing microbial susceptibility to antibiotics by treating hyper-resistant biofilms comprising gacS mutant variants with a gacS agonist the reverts the phenotype to a gacS wild-type. In one aspect, the hyper-resistant biofilms are treated with a combination of an antimicrobial agent and a gacS agonist.

According to the disclosure, biofilms prevented or treated by the disclosure can contain single species or multiple species bacteria. In one embodiment, the biofilms are associated with microbial infection (e.g., burns, wounds or skin ulcers) or a disease condition including, without limitation, dental caries, periodontal disease, prostatitis, osteomyelitis, septic arthritis, and cystic fibrosis.

In still another embodiment, the biofilms are associated with a surface, e.g., a solid surface. Such surface can be the surface of any industrial structure, e.g., pipeline or the surface of any structure in animals or humans. For example, such surface can be any epithelial surface, mucosal surface, or any host surface associated with bacterial infection, e.g., persistent and chronic bacterial infections. The surface can also include any surface of a bio-device in animals or humans, including without limitation, bio-implants such as bone prostheses, heart valves, and pacemakers.

In addition to surfaces associated with biofilm formation in a biological environment, the surfaces treated by the disclosure can also be any surface associated with industrial biofilm formation. For example, the surfaces being treated can be any surface associated with biofouling of pipelines, heat exchangers, air filtering devices, or contamination of computer chips or water-lines in surgical units like those associated with dental hand-pieces.

The term “purified” and “substantially purified” as used herein refers to a polypeptide or peptide that is substantially free of other proteins, lipids, and polynucleotides (e.g., cellular components with which an in vivo-produced polypeptide or peptide would naturally be associated). Typically, the peptide is at least 70%, 80%, or most commonly at least 90% pure by weight.

The term “gacS agonist” refers to a molecule that increases or decreases one or more gacS activities as does full-length native gacS. An example of a gacS agonist includes gacS polynucleotides capable of expression in a cell that replace or revert a mutant polynucleotide or phenotype associated a gacS mutant to a wild-type phenotype. Another example includes a gacS polypeptide capable of eliciting a wild-type activity in a null or mutant gacS phenotype. Other agonists include antibodies, peptides and small molecules. Various assays associated with gacS activity are known in the art and can be used to determine the activity of a gacS agonist.

The term “gacS antagonist” refers to a molecule that binds to a gacS polypeptide or polynucleotide and blocks or prevents the normal effect or expression, respectively, of gacS, thereby inhibiting the activity of a full length native gacS polypeptide or polynucleotide. Examples of gacS antagonists include inhibitory nucleic acid (e.g., antisense, ribozymes and the like), antibodies that bind and inhibit gacS and fragments of gacS that bind to gacS cognates and prevent interaction of a WT gacS with the cognate (e.g., such fragments include soluble fragments of gacS). Other agonists include antibodies, peptides and small molecules.

The term “gacA antagonist” refers to a molecule that binds to a gacA polypeptide or polynucleotide and blocks or prevents the normal effect or expression, respectively, of gacA, thereby inhibiting the activity of a full length native gacA polypeptide or polynucleotide. Examples of gacA antagonists include inhibitory nucleic acid (e.g., antisense, ribozymes and the like), antibodies to gacA and fragments of gacA that bind to gacA cognates (e.g., gacS) and prevent interaction of a WT gacA with the cognate (e.g., such fragments include soluble fragments of gacA).

A gacS polypeptide comprises a sequence as set forth in SEQ ID NO:2, and includes analogs, derivatives, conservative variations, and functional fragments of a gacS polypeptide capable of acting as a gacS agonist or antagonists. Such gacS analogs, derivatives, variants and fragments having agonist activities can be determined using the methods described herein. For example, a variant is an agonist if the variant can revert a mutant gacS phenotype to a wild-type phenotype. Such a reversion can be determined, for example, by measuring susceptible of a mutant (e.g., a SCV) P. aeruginosa or biofilm to an antimicrobial agent. It is not necessary that the analog, derivative, variation, or variant have activity identical to the activity of a wild-type gacS. In addition to Pseudomonas aeruginosa, many other organisms were also found to contain proteins bearing high levels of sequence identity to gacS.

In one aspect, a gacS polypeptide is an altered and/or truncated form of a wild-type gacS (e.g., SEQ ID NO:2). For example, an altered gacS polypeptide can comprise from about 1 to 10 amino acids substitution as compared to a reference wild-type gacS (e.g., SEQ ID NO:2). A “derivative” refers to a gacS polypeptide that comprises at least a portion of a biologically active gacS (including a gacS variant) and a second polypeptide or peptide. Derivatives can be produced by adding one or a few (e.g., 1-5) amino acids to a polypeptide of the disclosure without completely inhibiting the activity of the peptide. In addition, C-terminal derivatives, e.g., C-terminal methyl esters, can be produced and are encompassed by the disclosure.

The disclosure also includes gacS polypeptides that are conservative variations of a wild-type gacS polypeptide. The term “conservative variation” as used herein denotes a peptide or polypeptide in which at least one amino acid is replaced by another, biologically similar residue. Examples of conservative variations include the substitution of one hydrophobic residue, such as isoleucine, valine, leucine, alanine, cysteine, glycine, phenylalanine, proline, tryptophan, tyrosine, norleucine or methionine for another, or the substitution of one polar residue for another, such as the substitution of arginine for lysine, glutamic for aspartic acid, or glutamine for asparagine, and the like. Neutral hydrophilic amino acids that can be substituted for one another include asparagine, glutamine, serine and threonine. The term “conservative variation” also encompasses a peptide having a substituted amino acid in place of an unsubstituted parent amino acid; typically, antibodies raised to the substituted peptide or polypeptide also specifically bind the unsubstituted peptide or polypeptide.

A gacS polypeptide can comprise a peptide mimetic, which is a non-amino acid chemical structure that mimics the structure of, for example, a gacS polypeptide of SEQ ID NO:2, yet retains the ability to modulate gacA and/or revert a mutant gacS (e.g., a SCV phenotype) to a wild-type phenotype. Such a mimetic generally is characterized as exhibiting similar physical characteristics such as size, charge or hydrophobicity in the same spatial arrangement found in the gacS wild-type. A specific example of a peptide mimetic is a compound in which the amide bond between one or more of the amino acids is replaced, for example, by a carbon-carbon bond or other bond well known in the art (see, for example, Sawyer, Peptide Based Drug Design, ACS, Washington (1995)).

Typically a gacS polypeptide comprises the twenty naturally occurring amino acids, including, unless stated otherwise, L-amino acids and D-amino acids. The use of D-amino acids are particularly useful for increasing the life of a peptide or polypeptide. Polypeptides or peptides incorporating D-amino acids are resistant to proteolytic digestion. The term amino acid also refers to compounds such as chemically modified amino acids including amino acid analogs, naturally occurring amino acids that are not usually incorporated into proteins such as norleucine, and chemically synthesized compounds having properties known in the art to be characteristic of an amino acid, provided that the compound can be substituted within a peptide such that it retains its biological activity. Other examples of amino acids and amino acids analogs are listed in Gross and Meienhofer, The Peptides: Analysis, Synthesis, Biology, Academic Press, Inc., New York (1983). An amino acid also can be an amino acid mimetic, which is a structure that exhibits substantially the same spatial arrangement of functional groups as an amino acid but does not necessarily have both the “-amino” and “-carboxyl” groups characteristic of an amino acid.

A gacS polypeptide of the disclosure can comprise amino acids joined to each other by peptide bonds or modified peptide bonds, i.e., peptide isosteres, and may contain amino acids other than the 20 codon-encoded amino acids. The polypeptides may be modified by either natural processes, such as posttranslational processing, or by chemical modification techniques which are well known in the art. Such modifications are well described in basic texts and in more detailed monographs, as well as in a voluminous research literature. Modifications can occur anywhere in a peptide or polypeptide, including the peptide backbone, the amino acid side-chains and the amino or carboxyl termini. It will be appreciated that the same type of modification may be present in the same or varying degrees at several sites in a given peptide or polypeptide. Also, a given peptide or polypeptide may contain many types of modifications. A peptide or polypeptide may be branched, for example, as a result of ubiquitination, and they may be cyclic, with or without branching. Cyclic, branched, and branched cyclic peptides and polypeptides may result from posttranslation natural processes or may be made by synthetic methods. Modifications include acetylation, acylation, ADP-ribosylation, amidation, covalent attachment of flavin, covalent attachment of a heme moiety, covalent attachment of a nucleotide or nucleotide derivative, covalent attachment of a lipid or lipid derivative, covalent attachment of phosphotidylinositol, cross-linking, cyclization, disulfide bond formation, demethylation, formation of covalent cross-links, formation of cysteine, formation of pyroglutamate, formylation, gamma-carboxylation, glycosylation, GPI anchor formation, hydroxylation, iodination, methylation, myristoylation, oxidation, pegylation, proteolytic processing, phosphorylation, prenylation, racemization, selenoylation, sulfation, transfer-RNA mediated addition of amino acids to proteins such as arginylation, and ubiquitination. (See, for instance, PROTEINS—STRUCTURE AND MOLECULAR PROPERTIES, 2nd Ed., T. E. Creighton, W. H. Freeman and Company, New York (1993); POSTTRANSLATIONAL COVALENT MODIFICATION OF PROTEINS, B. C. Johnson, Ed., Academic Press, New York, pgs. 1-12 (1983); Seifter et al., Meth Enzymol 182:626-646 (1990); Rattan et al., Ann N.Y. Acad Sci 663:48-62 (1992).)

Peptides and polypeptides of the disclosure can be synthesized by commonly used methods such as those that include t-BOC or FMOC protection of alpha-amino groups. Both methods involve stepwise synthesis in which a single amino acid is added at each step starting from the C terminus of the peptide (See, Coligan, et al., Current Protocols in Immunology, Wiley Interscience, 1991, Unit 9). Peptides of the disclosure can also be synthesized by the well known solid phase peptide synthesis methods such as those described by Merrifield, J. Am. Chem. Soc., 85:2149, 1962; and Stewart and Young, Solid Phase Peptides Synthesis, Freeman, San Francisco, 1969, pp.27-62, using a copoly(styrene-divinylbenzene) containing 0.1-1.0 mMol amines/g polymer. On completion of chemical synthesis, the peptides can be deprotected and cleaved from the polymer by treatment with liquid HF-10% anisole for about ¼-1 hours at 0° C. After evaporation of the reagents, the peptides are extracted from the polymer with a 1% acetic acid solution, which is then lyophilized to yield the crude material. The peptides can be purified by such techniques as gel filtration on Sephadex G-15 using 5% acetic acid as a solvent. Lyophilization of appropriate fractions of the column eluate yield homogeneous peptide, which can then be characterized by standard techniques such as amino acid analysis, thin layer chromatography, high performance liquid chromatography, ultraviolet absorption spectroscopy, molar rotation, or measuring solubility. If desired, the peptides can be quantitated by the solid phase Edman degradation.

The disclosure also includes isolated polynucleotides (e.g., DNA, cDNA, or RNA) encoding a gacS polypeptide of the disclosure. Included are polynucleotides that encode analogs, mutants, conservative variations, and variants of the polypeptides described herein. The term “isolated” as used herein refers to a polynucleotide that is substantially free of proteins, lipids, and other polynucleotides with which an in vivo-produced polynucleotide naturally associates. Typically, the polynucleotide is at least 70%, 80%, and commonly at least 90% isolated from other matter. Conventional methods for synthesizing polynucleotides in vitro can be used in lieu of in vivo methods. As used herein, “polynucleotide” refers to a polymer of deoxyribonucleotides or ribonucleotides, in the form of a separate fragment or as a component of a larger genetic construct (e.g., by operably linking a promoter to a polynucleotide encoding a peptide of the disclosure). Numerous genetic constructs (e.g., plasmids and other expression vectors) are known in the art and can be used to produce a polypeptide of the disclosure in cell-free systems or prokaryotic or eukaryotic (e.g., yeast, insect, or mammalian) cells. By taking into account the degeneracy of the genetic code, one of ordinary skill in the art can readily synthesize polynucleotides encoding the polypeptides of the disclosure. The polynucleotides of the disclosure can readily be used in conventional molecular biology methods to produce the peptides of the disclosure.

In one embodiment, a gacS polynucleotide of the disclosure comprises a sequence of SEQ ID NO:1. The gacS polynucleotide comprises an export signal with a predicted cleavage site after either amino acid 23 or 27 of SEQ ID NO:2. Accordingly, in one aspect, a polynucleotide encoding a gacS polypeptide comprises SEQ ID NO:1 from about nucleotide 70 or 82 to about 2774. In addition, the gacS polypeptide comprises a number of putative hydrophobic or transmembrane domains. Thus, the disclosure further contemplates the use of soluble polypeptides and polynucleotides encoding the soluble fragments of gacS. For example, putative transmembrane domains comprise amino acids 10-30, 167-188, 576-585, and 826-836 of SEQ ID NO:2 (one of skill in the art can ascertain the corresponding polynucleotide sequences from the sequences listing appended hereto).

| | |
|---|---|
| GTGTTCAAGGATCTCGGCATCAAGGGGCGCGTACTGCTGCTCACCCTGCTCCCCACCAGCCTGCTGGCGATGGT | (SEQ ID NO: 1) |
| GCTTGGCGGTTACTTCACCTGGGTCCAGCTGTCCGACATGCGCGCCCAGTTGATCGAGCGCGGGCAACTGATCG | |
| CCGAACAACTGGCGCCGCTGGCCGCCACCGCGCTGGCGCGAAAGGATACCGCCGTGCTCAACCGCATCGCCAAC | |
| GAGGCGCTGGACCAACCGGACGTGCGCGCGGTGACCTTCCTCGACGCCCGCCAGGAACGCCTCGCCCATGCCGG | |
| GCCAAGCATGCTCACCGTCGCCCCGGCCGGCGACGCCAGCCATTTGAGCATGTCCACCGAACTGGACACCACGC | |
| ACTTCCTGCTACCGGTTCTTGGCCGCCACCACAGCCTGTCCGGCGCCACCGAGCCTGACGACGAGCGCGTACTC | |
| GGCTGGGTCGAACTGGAACTGTCGCACCACGGGACTCTGCTGCGCGGATATCGCAGCCTGTTCACCAGCCTCTT | |
| GCTGATCGCCGCCGGCCTCGGCGTCACCGCCCTCCTCGCCCTGCGCATGAGCCGCGCGATCAACGCGCCGCTGG | |
| AACTGATCAGCCAGGGCGTCGCCCAGCTCAAGGAAGGCCGCATGGAAACCCGCCTGCCACCGATGGGCAGCAAC | |
| GAGCTGGACGAACTGGCCTCTGGCATCAACCGCATGGCGGAAACGCTGCAGAGCGCCCAGGAGGAAATGCAGCA | |
| CAACATCGACCAGGCCACCGAGGACGTACGGCAGAACCTGGAAACCATCGAGATCCAGAACATCGAGCTGGACC | |
| TGGCGCGCAAGGAGGCCCTGGAGGCGAGCAGGATCAAGTCCGAGTTCCTCGCCAACATGAGCCACGAGATCCGC | |
| ACCCCGCTCAACGGCATCCTCGGTTTCACCAACCTGCTGCAGAAGAGCGAGCTCAGCCCGCGCCAGCAGGACTA | |
| CCTCACGACCATCCAGAAATCGGCGGAAAGCCTGCTGGGGATCATCAACGAGATCCTCGATTTCTCGAAGATCG | |
| AGGCCGGCAAGCTGGTTCTGGAAAACCTCCCTTTCAATCTCCGCGACCTGATCCAGGACGCCCTGACCATGCTG | |
| GCTCCGGCCGCCCACGAGAAGCAACTGGAACTGGTCAGCCTGGTCTACCGGGATACCCCGATCCAATTGCAGGG | |
| CGACCCGCAGCGGCTGAAGCAGATCCTCACCAACCTGGTCGGCAACGCCATCAAGTTCACCCAGGGCGGCACCG | |
| TCGCCGTACGCGCCATGCTCGAGGACGAAAGCGACGACCGCGCGCAGCTGCGGATCAGCGTCCAGGACACCGGT | |
| ATCGGCCTCTCCGAGGAAGACCAGCAAGCCTTGTTCAAGGCCTTCAGCCAGGCCGACAACTCACTGTCGCGGCA | |
| AGCCGGTGGCACCGGCCTGGGCCTGGTGATCTCCAAGCGCCTGATTGAGCAGATGGGCGGCGAGATCGGCGTCG | |
| ACAGTACGCCTGGGGAAGGCGCCGAGTTCTGGATCAGCCTGAGTCTGCCGAAAAGTCGCGACGACAACGAGGAG | |
| CCGGGCGCCTCCTGGGCCGCGGGCCAACGCGTGGCGCTGCTCGAACCGCAGGAACTGACGCGCCGCTCGCTGCA | |
| CCACCAGCTCACCGACTTCGGCCTGGAAGTGAGCGAATTCGCCGACCTCGACAGCCTCCAGGAAAGCCTGCGCA | |
| ACCCGCCGCCCGGCCAGTTGCCGATCAGCCTGGCGGTGCTCGGCGTCTCGGCCGCGATCCATCCGCCGGAAGAG | |
| CTGAGCCAGTCGTTCTGGGAATTCGAACGGCTCGGCTGCAAGACCCTGGTGCTCTGCCCGACCACCGAGCAGGC | |
| GCAATACCACGCGACCCTGCCCGACGAACAGGTCGAGGCCAAGCCCGCCTGCACCCGCAAGCTGCAACGCAAGC | |
| TGCAGGAGTTGCTTCAAGTCCGCCCGACGCGCAGCGACAAGCCCCACGCCATGGTTTCCGGACGGCCGCCACGG | |
| CTGCTATGCGTCGACGACAACCCGGCCAACCTGCTGCTGGTGCAGACCCTGCTCAGCGACCTCGGCGCCCAGGT | |
| CACCGCGGTGGACAGCGGCTACGCGGCCCTCGAGGTAGTGCAGCGCGAGCGCTTCGACCTGGTCTTCATGGACG | |
| TGCAGATGCCCGGCATGGACGGCCGCCAGGCCACCGAGGCGATCCGCCGCTGGGAGGCCGAGCGGGAAGTCAGC | |
| CCGGTGCCGGTGATCGCGCTCACCGCACATGCGCTTTCCAACGAGAAGCGCGCATTGCTGCAGGCCGGCATGGA | |
| CGACTACCTGACCAAGCCGATCGACGAGCAGCAATTGGCCCAGGTAGTGCTGAAGTGGACCGGACTGAGCCTGG | |
| GCCAGTCGCTGGCCAGCATGAGCCGTGCGCCGCAGCTCGGCCAGTTGAGCGTGCTCGACCCCGAGGAAGGGCTG | |
| CGCCTGGCCGCCGGCAAGGCCGACCTCGCCGCCGACATGCTGGCGATGCTGCTGGCCTCGCTGGCGGCGGACCG | |
| CCAGGCGATTCGCCAGGCCCGCGACAACGACGACCGCACCGCTTTGCTCGAGAGGGTCCACCGGCTGCATGGCG | |
| CCACCCGCTACTGTGGCGTGCCGCAGTTGCGCGCGGCCTGCCAGACCAGCGAAACCCTGCTCAAGCAGAACGAT | |
| CCGGCGGCGGCCGCGGCCCTGGACGAGCTGGACAAGGCCATCGAGGCCCTGGCCGACACTGCCTCGGCCACCAC | |
| CCACCTGTCCTCCACCAGCCTCGACTCCAGCGAACTCTGA | |
| MFKDLGIKGRVLLLTLLPTSLLAMVLGGYFTWVQLSDMRAQLIERGQLIAEQLAPLAATALARKDTAVLNRIAN | (SEQ ID NO: 2) |
| EALDQPDVRAVTFLDARQERLAHAGPSMLTVAPAGDASHLSMSTELDTTHFLLPVLGRHHSLSGATEPDDERVL | |
| GWVELELSHHGTLLRGYRSLFTSLLLIAAGLGVTALLALRMSRAINAPLELISQGVAQLKEGRMETRLPPMGSN | |
| ELDELASGINRMAETLQSAQEEMQHNIDQATEDVRQNLETIEIQNIELDLARKEALEASRIKSEFLANMSHEIR | |
| TPLNGILGFTNLLQKSELSPRQQDYLTTIQKSAESLLGIINEILDFSKIEAGKLVLENLPFNLRDLIQDALTML | |
| APAAHEKQLELVSLVYRDTPIQLQGDPQRLKQILTNLVGNAIKFTQGGTVAVRAMLEDESDDPAQLRISVQDTG | |
| IGLSEEDQQALFKAFSQADNSLSRQAGGTGLGLVISKRLIEQMGGEIGVDSTPGEGAEFWISLSLPKSRDDNEE | |
| PGASWAAGQRVALLEPQELTRRSLHHQLTDFGLEVSEFADLDSLQESLRNPPPGQLPISLAVLGVSAAIHPPEE | |
| LSQSFWEFERLGCKTLVLCPTTEQAQYHATLPDEQVEAKPACTRKLQRKLQELLQVRPTRSDKPHAMVSGRPPR | |
| LLCVDDNPANLLLVQTLLSDLGAQVTAVDSGYAALEVVQRERFDLVFMDVQMPGMDGRQATEAIRRWEAEREVS | |
| PVPVIALTAHALSNEKRALLQAGMDDYLTKPIDEQQLAQVVLKWTGLSLGQSLASMSRAPQLGQLSVLDPEEGL | |
| RLAAGKADLAADMLAMLLASLAADRQAIRQARDNDDRTALLERVHRLHGATRYCGVPQLRAACQTSETLLKQND | |
| PAAAAALDELDKAIEALADTASATTHLSSTSLDSSEL* | |
| (* denotes stop codon) | |

Such polynucleotides include naturally occurring, synthetic, and intentionally manipulated polynucleotides. For example, a gacS polynucleotide may be subjected to site-directed mutagenesis. A gacS polynucleotide includes sequences that are degenerate as a result of the genetic code. There are 20 natural amino acids, most of which are specified by more than one codon. Therefore, all degenerate nucleotide sequences are included so long as the amino acid sequence of a gacS polypeptide encoded by the polynucleotide is functionally unchanged. Accordingly, a polynucleotide of the invention includes (i) a polynucleotide encoding a gacS polypeptide; (ii) a polynucleotide encoding SEQ ID NO:2 or a variant thereof comprising a gacS agonist or antagonist activity; (iii) a polynucleotide comprising SEQ ID NO:1; (iv) a polynucleotide of (i-iii), wherein T is U; and (v) a polynucleotide comprising a sequence that is complementary to (iii) and (iv) above. Polynucleotides capable of hybridizing, under stringent hybridization conditions, to a polynucleotide consisting of SEQ ID NO:1 or fragment thereof and encoding a gacS polypeptide (e.g., SEQ ID NO:2 or fragment thereof) are also contemplated by the disclosure. “Stringent hybridization conditions” refers to an overnight incubation at 42° C. in a solution comprising 50% formamide, 5×SSC (750 mM NaCl, 75 mM trisodium citrate), 50 mM sodium phosphate (pH 7.6), 5× Denhardt's solution, 10% dextran sulfate, and 20 μg/ml denatured, sheared salmon sperm DNA, followed by washing the filters in 0.1×SSC at about 65° C. It will be recognized that a polynucleotide of the disclosure, may be operably linked to a second heterologous polynucleotide such as a promoter or a heterologous sequence encoding a desired peptide or polypeptide sequence.

Polynucleotides encoding the gacS polypeptide of the disclosure can be inserted into an “expression vector.” The term “expression vector” refers to a genetic construct such as a plasmid, virus or other vehicle known in the art that can be engineered to contain a polynucleotide encoding a peptide or polypeptide of the disclosure. Such expression vectors are typically plasmids that contain a promoter sequence that facilitates transcription of the inserted genetic sequence in a host cell. The expression vector typically contains an origin of replication, and a promoter, as well as genes that allow phenotypic selection of the transformed cells (e.g., an antibiotic resistance gene). Various promoters, including inducible and constitutive promoters, can be utilized in the disclosure. Typically, the expression vector contains a replicon site and control sequences that are derived from a species compatible with the host cell.

Transformation or transfection of a host cell with a polynucleotide of the disclosure can be carried out using conventional techniques well known to those skilled in the art. For example, DNA uptake can be facilitated using the CaCl₂, MgCl₂ or RbCl methods known in the art. Alternatively, physical means, such as electroporation or microinjection can be used. Electroporation allows transfer of a polynucleotide into a cell by high voltage electric impulse. Additionally, polynucleotides can be introduced into host cells by protoplast fusion, using methods well known in the art. Naked DNA can be used (e.g., naked plasmid DNA). Yet in another aspect, bacteriophage can be used to deliver a gacS polynucleotide to a bacteria or bacterial biofilm.

“Host cells” encompassed by of the disclosure are any cells in which the polynucleotides of the disclosure can be used to express the gacS polypeptides of the disclosure. The term also includes any progeny of a host cell. Introduction of the construct into the host cell can be effected by calcium phosphate transfection, DEAE-Dextran mediated transfection, or electroporation (Davis, L., Dibner, M., Battey, I., Basic Methods in Molecular Biology (1986)).

Polynucleotides encoding the polypeptides of the disclosure can be isolated from a cell (e.g., a cultured cell), or they can be produced in vitro. A polynucleotide encoding a gacS polypeptide can be obtained by: 1) isolation of a double-stranded DNA sequence from genomic DNA; 2) chemical manufacture of a polynucleotide such that it encodes the gacS polypeptide; or 3) in vitro synthesis of a double-stranded DNA sequence by reverse transcription of RNA isolated from a donor cell (i.e., to produce cDNA).

Any of various art-known methods for protein purification can be used to isolate the polypeptides of the disclosure. For example, preparative chromatographic separations and immunological separations (such as those employing monoclonal or polyclonal antibodies) can be used. Carrier peptides can facilitate isolation of fusion proteins that include the peptides of the disclosure. Purification tags can be operably linked to a gacS polypeptide of the disclosure. For example, glutathione-S-transferase (GST) allows purification with a glutathione agarose affinity column. When either Protein A or the ZZ domain from Staphylococcus aureus is used as the tag, purification can be accomplished in a single step using an IgG-sepharose affinity column. For example, monoclonal or polyclonal antibodies that specifically bind the gacS polypeptide can be used in conventional purification methods. Techniques for producing such antibodies are well known in the art.

A fusion construct comprising a peptide or polypeptide linked to a gacS polypeptide can be linked at either the amino or carboxy terminus of the peptide. Typically, the polypeptide that is linked to the gacS polypeptide is sufficiently anionic or cationic such that the charge associated with the gacS polypeptide is overcome and the resulting fusion peptide has a net charge that is neutral or negative. The peptide or polypeptide linked to a gacS polypeptide can correspond in sequence to a naturally-occurring protein or can be entirely artificial in design. Functionally, the polypeptide linked to a gacS polypeptide (the “carrier polypeptide”) may help stabilize the gacS polypeptide and protect it from proteases, although the carrier polypeptide need not be shown to serve such a purpose. Similarly, the carrier polypeptide may facilitate transport of the fusion polypeptide. Examples of carrier polypeptides that can be utilized include anionic pre-pro peptides and anionic outer membrane peptides. Examples of carrier polypeptides include glutathione-S-transferase (GST), protein A of Staphylococcus aureus, two synthetic IgG-binding domains (ZZ) of protein A, outer membrane protein F of Pseudomonas aeruginosa, and the like. The disclosure is not limited to the use of these polypeptides; others suitable carrier polypeptides are known to those skilled in the art. In another aspect, a linker moiety comprising a protease cleavage site may be operably linked to a gacS polypeptide of the disclosure. For example, the linker may be operable between to domains of a fusion protein (e.g., a fusion protein comprising a gacS polypeptide and a carrier polypeptide). Because protease cleavage recognition sequences generally are only a few amino acids in length, the linker moiety can include the recognition sequence within flexible spacer amino acid sequences, such as GGGGS (SEQ ID NO:3). For example, a linker moiety including a cleavage recognition sequence for Adenovirus endopeptidase could have the sequence GGGGGGSMFG GAKKRSGGGG GG (SEQ ID NO:4). If desired, the spacer DNA sequence can encode a protein recognition site for cleavage of the carrier polypeptide from the gacS polypeptide. Examples of such spacer DNA sequences include, but are not limited to, protease cleavage sequences, such as that for Factor Xa protease, the methionine, tryptophan and glutamic acid codon sequences, and the pre-pro defensin sequence. Factor Xa is used for proteolytic cleavage at the Factor Xa protease cleavage sequence, while chemical cleavage by cyanogen bromide treatment releases the peptide at the methionine or related codons. In addition, the fused product can be cleaved by insertion of a codon for tryptophan (cleavable by o-iodosobenzoic acid) or glutamic acid (cleavable by Staphylococcus protease). Insertion of such spacer oligonucleotides is not a requirement for the production of gacS polypeptides, such oligonucleotide can enhance the stability of the fusion polypeptide.

As depicted in FIG. 17, the disclosure demonstrates that inhibiting the activity of the gacS/gacA two component system inhibits the formation biofilms. In addition, contacting hyper-resistant biofilms (generated through a process of gacS mutations via stress and age) with an agent that promotes a wild-type gacS phenotype reverts small colony variants (SCV) and renders them susceptible to antimicrobials thereby reducing biofilm formation. Such SCV comprise genomes that have mutated and in many cases result in resistant microbes and hyper-resistant biofilms. By inhibiting gacS in the wild-type bacterial species, the production of biofilms are reduced. Accordingly, the disclosure provides methods of inhibiting the formation of biofilms comprising inhibiting the activity or production of gacS or the activity or production of gacA using a gacS antagonist and/or gacA antagonist. The disclosure demonstrates that gacS knockout bacterial were unable to form biofilms. The gacS knock-out mutant was deficient in phenazine, acyl homoserine lactones and extracellular protease production. The ability of wild type and mutant strains to form biofilms was evaluated in vitro using the MBEC device (as described more fully below). Biofilm formation by the gacS mutant, as evaluated by colony counts and SEM was greatly reduced, but it was restored by complementation with an active gacS construct. The results demonstrate that the regulatory gacS gene plays an important role in biofilm formation and structure in PcO6, which may play a role in its biocontrol capability.

In one embodiment, the disclosure is directed to methods of inhibition of biofilm formation by pathogenic bacteria. It is contemplated that inhibitors, antagonists or antibodies of the GacA/GacS regulatory system can also be used to inhibit biofilm formation of, and to treat diseases associated with, biofilm formation. Proteins which are homologous to gacS and gacA and the organisms which contain these proteins can be found by sequence homology searches known in the art. In particular, the following are examples of proteins which have a sequence identity of at least 25% with GacA:

| | | Sequence |
|---|---|---|
| Organism | Protein | Identity |
| Pseudomonas aeruginosa | GacA | 100% |
| P. viridiflava | RepB | 89% |
| P. syringae | cognate response regulator | 89% |
| | gacA | |
| P. syringae | fix J-like response regulator | 89% |
| P. fluorescense | response regulator (AF065156) | 87% |
| P. fluorescense | response regulator/ | 86% |
| | transcription activator | |
| | (L29642) | |
| P. fluorescense | gacA (M80913) | 86% |
| V. cholerae | transcription regulator luxR | 62% |
| | family | |
| E. coli 0157:H7 | | 60% |
| E. coli | UVRY protein | 60% |
| Salmonella Typhimurium | SirA | 60% |
| Erwinia carotovora | expA | 59% |
| Xylella fasticliosci | luxR/uhpA | 43% |
| Streptomyces coe | two-component response | 40% |
| | regulator | |
| Deinococcus radiodurans | | 38% |
| P. Solonacearum | vsrD protein | 37% |
| Ralstonia solanacearum | vsrD protein | 37% |
| V. cholerae | transcription regulator LuxR | 37% |
| | family VC1277 | |
| P. aeruginosa | nitrate/nitrite regulatory | 36% |
| | protein | |
| P. aeruginosa | two-component response | 36% |
| | regulator NarL | |
| Streptomyces coelicolor | A3(2) (AL355774) | 36% |
| Neisse meningitidis | transcriptional regulator, | 34% |
| | LuxR family | |
| Deinococus radiodurans | DNA-binding response | 34% |
| | regulator | |
| P. aeruginosa | two-component response | 34% |
| | regulator PA3045 | |
| Streptomyces coelicolor | putative response regulator | 34% |
| Streptococcus pneumoniae | response regulator | 32% |
| S. coelicolor | A3(2) (AL049754) | 33% |
| B. subtilis [yvqe] | homolog yvqc | 32% |
| Bacillus h. | two-component regulator | 33% |
| P. aeruginosa | two-component regulator | 34% |
| | PA0601 | |
| Streptomyces coelicolor | A3 | 34% |
| Lactococcus lactis | RrD | 34% |
| Synechocystis sp. | nitrate/nitrite response | 34% |
| | regulator protein | |
| Streptomyces coelicolor | response regulator | 32% |
| Bordetella pertussis | bvgA | 34% |
| Bordetella bronchiseptica | bvgA | 34% |
| Bordetella parapertussis | bvgA | 34% |
| Erwinia amy | HrpY | 30% |
| Staphylococcus aureus | response regulator | 31% |
| Deinococcus radiodurans | DNA-binding response regulator | 33% |
| P. Stutzeri | NarL protein | 32% |
| Bacillus h. | response regulator | 31% |
| Bacallus subtilis | yfik | 29% |
| Bacillus brevis | DEGU regulatory protein | 27% |
| Bacillus halodurans | two-component response | 27% |
| | regulator | |
| Bacillus subtilis | DEGU, extracellular proteinase | 26% |
| | response regulator | |

In another embodiment, the disclosure provides methods of regulation of biofilm formation by symbiotic bacteria, for example, plant root bacteria. It is contemplated that activators, inhibitors, agonists, antagonists or antibodies of the GacA/GacS regulatory system can also be used to regulate biofilm formation. For example, Pseudomonas chlororaphis O6 (PcO6) is an aggressive colonizer of plant roots under competitive soil conditions. Root colonization by PcO6 induces foliar resistance to Pseudomonas syringae pv. tabaci in tobacco. The disclosure demonstrates that gacS knockout bacterial were unable to form biofilms. The gacS knock-out mutant was deficient in phenazine, acyl homoserine lactones and extracellular protease production. The ability of wild type and mutant strains to form biofilms was evaluated in vitro using the MBEC device (as described more fully below). Biofilm formation by the gacS mutant, as evaluated by colony counts and SEM was greatly reduced, but it was restored by complementation with an active gacS construct. The results demonstrate that the regulatory gacS gene plays an important role in biofilm formation and structure in PcO6, which may play a role in its biocontrol capability.

Accordingly, the disclosure provides a method of inhibiting biofilm or SCV resistant phenotypes comprising inhibiting the gacS/gacA system. In one aspect, inhibition of gacS is performed by contacting a bacteria or biofilm with an agent (e.g., a gacS and/or gacA antagonist) that inhibits gacS and/or gacA activity or production. For example, an agent useful for inhibiting gacS activity comprises an antibody. The antibody specifically binds to gacS. In another aspect, the disclosure provides inhibitory polynucleotides (e.g., ribozymes, antisense and/or siRNA) that specifically binds to a polynucleotide encoding a gacS or a gacA polypeptide. For example, the inhibitory polynucleotides interact with a polynucleotide consisting of a sequence as set forth in SEQ ID NO:1 preventing its transcription or translation.

Ribozymes are catalytically active nucleic acids which consist of RNA which basically comprises two moieties. The first moiety shows a catalytic activity whereas the second moiety is responsible for the specific interaction with the target nucleic acid (e.g., a gacS polynucleotide). Upon interaction between the target nucleic acid and the second moiety of the ribozyme, typically by hybridisation and Watson-Crick base pairing of essentially complementary stretches of bases on the two hybridising strands, the catalytically active moiety may become active which means that it catalyses, either intramolecularly or intermolecularly, the target nucleic acid in case the catalytic activity of the ribozyme is a phosphodiesterase activity. Subsequently, there may be a further degradation of the target nucleic acid which in the end results in the degradation of the target nucleic acid as well as the protein derived from the said target nucleic acid. Ribozymes, their use and design principles are known to the one skilled in the art, and, for example described in Doherty, E. et al., 2001, and Lewin, A. et al., 2001.

The activity and design of antisense oligonucleotides for the manufacture of a medicament is based on a similar mode of action. Basically, antisense oligonucleotides hybridize based on base complementarity, with a target RNA. When the antisense molecule hybridizes with the target polynucleotide the double stranded RNA-DNA or RNA-RNA molecule become susceptible to RNAse H activity which degrades double stranded RNA. Alternatively, the double stranded molecule prevents translation of the target polynucleotide.

Furthermore, the disclosure demonstrates that as part of a stress response WT bacteria generate mutant gacS variants. Resistant SCV's comprise mutant gacS genes, which when contacted with an agonist of gacS increases SCV antibiotic susceptibility. Accordingly, the disclosure provides methods and compositions useful for rendering SCV resistant biofilms susceptible to antibiotics. Agonist include wild-type gacS, polynucleotides encoding a wild-type gacS and the like.

The disclosure provides compositions useful for treating biofilm formation and infections associated with biofilm formation. The composition of the disclosure contains either a gacS/gacA antagonist or a gacS agonist, depending upon the resistance phenotype and stage of the biofilm. For example, where resistant SCVs have not formed a gacS and/or gacA antagonist or inhibitor is useful to prevent biofilm formation; however, where resistant SCVs have formed contacting the SCV resistant biofilms with an agonist is useful for rendering the bacterial susceptible to an antimicrobial or inhibiting antibacterial resistance.

Compositions useful in the methods of the disclosure can comprise a gacS antagonist or agonist (depending upon the phenotype) in combination with an antimicrobial agent. Such antimicrobial agent include, without limitation, detergents, penicillin, quinoline, vancomycin, sulfonamide, ampicillin, ciprofloxacin, sulfisoxazole, and biocides including chlorine or dose detergent.

The composition or agent of the disclosure can also include one or more other non-active ingredients, e.g., ingredients that do not interfere with the function of the active ingredients. For example, the composition or agent of the disclosure can include a suitable carrier or be combined with other therapeutic agents.

In one aspect, biofilm formation is prevented by contacting a bacteria in a biofilm with a composition comprising a gacS inhibitor (e.g., an antibody, a small molecule, a polypeptide such as a soluble fragment that inhibits gacS-gacA interaction, and/or inhibitory nucleic acids) under conditions and in a formulation that allows the gacS inhibitor to interact with the bacteria. Small colony variants are formed form wild-type gacS⁺ cells but these gacS⁺ SCVs are not stable. Stable SCV are derived from gacS⁻ cells. Accordingly, the methods of treatment are based upon a continuum of genetic modifications that lead to hyper-resistant biofilms. Modifications at early stages in biofilm formation (e.g., prior to stable SCV formation) utilize gacS antagonists; however, later biofilm (e.g., stable SCV and gacS⁻ hyper-resistant biofilms) comprise treatments that utilize promote a gacS⁺ wild-type phenotype.

In yet another aspect, biofilm formation is prevented by contacting a SCV bacteria in a biofilm with a composition comprising a gacS agonist (e.g., an antibody, a small molecule, a polypeptide such as a soluble fragment that increases or activates gacS, and/or inhibitory nucleic acids) under conditions and in a formulation that allows the gacS agonist to interact with the SCV bacteria.

In a further aspect, a combination therapy may be used. In this aspect, biofilm formation is inhibited by contacting a bacteria in a hyper-resistant biofilm with a gacS agonist and an antimicrobial agent.

The term “contacting” refers to exposing the bacterium to a gacS agonist or inhibitor so that the gacS agonist or inhibitor can modulate gacS activity or production thereby modulating the ability of the bacterial to generate SCVs or render SCV susceptible to antimicrobial agents. Contacting of an organism with a gacS agonist or inhibitor of the disclosure can occur in vitro, for example, by adding the agonist or inhibitor to a bacterial culture to test for susceptibility of the bacteria to the agonist or inhibitor, or contacting a bacterially contaminated surface with the agonist or inhibitor. Alternatively, contacting can occur in vivo, for example by administering the agonist or inhibitor to a subject afflicted with a bacterial infection or susceptible to infection. In vivo contacting includes both parenteral as well as topical.

“Inhibiting” or “inhibiting effective amount” refers to the amount of agonist or inhibitor that is sufficient to cause, for example, antimicrobial susceptibility, or a bacteriostatic or bactericidal effect, respectively. Bacteria that can be affected by the gacS agonist and inhibitors of the disclosure include both gram-negative and gram-positive bacteria. For example, bacteria that can be affected include Staphylococcus aureus, Streptococcus pyogenes (group A), Streptococcus sp. (viridans group), Streptococcus agalactiae (group B), S. bovis, Streptococcus (anaerobic species), Streptococcus pneumoniae, and Enterococcus sp.; Gram-negative cocci such as, for example, Neisseria gonorrhoeae, Neisseria meningitidis, and Branhamella catarrhalis; Gram-positive bacilli such as Bacillus anthracis, Bacillus subtilis, P.acne Corynebacterium diphtheriae and Corynebacterium species which are diptheroids (aerobic and anerobic), Listeria monocytogenes, Clostridium tetani, Clostridium difficile, Escherichia coli, Enterobacter species, Proteus mirablis and other sp., Pseudomonas aeruginosa, Klebsiella pneumoniae, Salmonella, Shigella, Serratia, and Campylobacter jejuni. Infection with one or more of these bacteria can result in diseases such as bacteremia, pneumonia, meningitis, osteomyelitis, endocarditis, sinusitis, arthritis, urinary tract infections, tetanus, gangrene, colitis, acute gastroenteritis, impetigo, acne, acne posacue, wound infections, born infections, fascitis, bronchitis, and a variety of abscesses, nosocomial infections, and opportunistic infections.

Fungal organisms may also be affected by the gacS polypeptides of the disclosure and include dermatophytes (e.g., Microsporum canis and other Microsporum sp.; and Trichophyton sp. such as T. rubrum, and T. mentagrophytes), yeasts (e.g., Candida albicans, C. Tropicalis, or other Candida species), Saccharomyces cerevisiae, Torulopsis glabrata, Epidermophyton floccosum, Malassezia furfur (Pityropsporon orbiculare, or P. ovale), Cryptococcus neoformans, Aspergillus fumigatus, Aspergillus nidulans, and other Aspergillus sp., Zygomycetes (e.g., Rhizopus, Mucor), Paracoccidioides brasiliensis, Blastomyces dermatitides, Histoplasma capsulatum, Coccidioides immitis, and Sporothrix schenckii.

An agonist or antagonist of the disclosure can be administered to any host, including a human or non-human animal, in an amount effective to inhibit growth of a bacterium and/or fungus. Thus, the agonist and antagonist are useful as antimicrobial agents and/or antifungal agents.

Any of a variety of art-known methods can be used to administer the agonist or antagonist to a subject. For example, the agonist or antagonist of the disclosure can be administered parenterally by injection or by gradual infusion over time. The peptide can be administered intravenously, intraperitoneally, intramuscularly, subcutaneously, intracavity, by inhalation, or transdermally.

In another aspect, a gacS agonist or antagonist of the disclosure may be formulated for topical administration (e.g., as a lotion, cream, spray, gel, or ointment). Such topical formulations are useful in treating or inhibiting microbial or fungal presence or infections on bio-devices, contaminated surfaces, the eye, skin, and mucous membranes such as mouth, vagina and the like. Examples of formulations in the market place include topical lotions, creams, soaps, wipes, and the like. It may be formulated into liposomes to reduce toxicity or increase bioavailability. Other methods for delivery of the agonist or antagonist include oral methods that entail encapsulation of the polypeptide or peptide in microspheres or proteinoids, aerosol delivery (e.g., to the lungs), or transdermal delivery (e.g., by iontophoresis or transdermal electroporation). Other methods of administration will be known to those skilled in the art.

Preparations for parenteral administration of an agonist or antagonist of the disclosure include sterile aqueous or non-aqueous solutions, suspensions, and emulsions. Examples of non-aqueous solvents are propylene glycol, polyethylene glycol, vegetable oils (e.g., olive oil), and injectable organic esters such as ethyl oleate. Examples of aqueous carriers include water, saline, and buffered media, alcoholic/aqueous solutions, and emulsions or suspensions. Examples of parenteral vehicles include sodium chloride solution, Ringer's dextrose, dextrose and sodium chloride, lactated Ringer's, and fixed oils. Intravenous vehicles include fluid and nutrient replenishers, electrolyte replenishers (such as those based on Ringer's dextrose), and the like. Preservatives and other additives such as, other antimicrobial, anti-oxidants, cheating agents, inert gases and the like also can be included.

The disclosure provides a method for inhibiting a topical bacterial and/or fungal-associated disorder by contacting or administering a therapeutically effective amount of an agonist or antagonist to a subject who has, or is at risk of having, such a disorder. Examples of disease signs that can be ameliorated include an increase in a subject's blood level of TNF, fever, hypotension, neutropenia, leukopenia, thrombocytopenia, disseminated intravascular coagulation, adult respiratory distress syndrome, shock, and organ failure. Examples of subjects who can be treated in the disclosure include those at risk for, or those suffering from, a toxemia, such as endotoxemia resulting from a gram-negative bacterial infection. Other examples include subjects having dermatitis as well as those having skin infections or injuries subject to infection with gram-positive or gram-negative bacteria or a fungus. Examples of candidate subjects include those suffering from infection by E. coli, Hemophilus influenza B, Neisseria meningitides, staphylococci, or pneumococci. Other patients include those suffering from gunshot wounds, renal or hepatic failure, trauma, burns, immunocompromising infections, hematopoietic neoplasias, multiple myeloma, Castleman's disease or cardiac myxoma. Those skilled in the art of medicine can readily employ conventional criteria to identify appropriate subjects for treatment in accordance with the disclosure.

The term “therapeutically effective amount” as used herein for treatment of a subject afflicted with a disease or disorder means an amount of gacS agonist or antagonist sufficient to ameliorate a sign or symptom of the disease or disorder or the presence of a biofilm. For example, a therapeutically effective amount can be measured as the amount sufficient to decrease the number or size of a biofilm, a subject's symptoms or rash by measuring the frequency of severity of skin sores etc. Typically, the subject is treated with an amount of gacS agonist or antagonist sufficient to reduce biofilm formation, decrease the number of SCVs formed, or the susceptibility of bacteria to an antimicrobial agent.

If desired, a suitable therapy regime can combine administration of an agonist or antagonist with one or more additional therapeutic agents (e.g., an inhibitor of TNF, an antibiotic, and the like). The agonist or antagonist, other therapeutic agents, and/or antibiotic(s) can be administered, simultaneously, but may also be administered sequentially. Suitable antibiotics include aminoglycosides (e.g., gentamicin), beta-lactams (e.g., penicillins and cephalosporins), quinolones (e.g., ciprofloxacin), and novobiocin. Generally, the antibiotic is administered in a bactericidal, antiviral and/or antifungal amount. The peptide provides for a method of increasing antibiotic activity by permeabilizing the bacterial outer membrane and combinations involving peptide and a sub-inhibitory amount (e.g., an amount lower than the bactericidal amount) of antibiotic can be administered. Typically, the gacS agonist or antagonist and antibiotic are administered within 48 hours of each other (e.g., 2-8 hours, or may be administered simultaneously). A “bactericidal amount” is an amount sufficient to achieve a bacteria-killing concentration. In accordance with its conventional definition, an “antibiotic,” as used herein, is a chemical substance that, in dilute solutions, inhibits the growth of, or kills microorganisms. Also encompassed by this term are synthetic antibiotics (e.g., analogs) known in the art. In another aspect, a method or composition disclosure may further comprise a divalent or monovalent metal chelator.

The following examples are intended to illustrate but not limit the disclosure or the appended claims.

### EXAMPLES

### Example 1

Growth conditions of Pseudomonas chlororaphis O6. Pseudomonas chlororaphis O6 wild type strain was isolated from roots of wheat plants grown in Logan, Utah, USA. P. chlororaphis O6 knockout gacS mutant strain and gacS complemented strain were generated. Bacteria were grown in 5.0 mL of King's medium (KB) (Protease peptone #3(Difco)-20 g, KH₂PO₄-1.5 g, MgSO₄-7H₂O-1.5 g, Glycerol-15.0 mL per L) at room temperature (18-22° C.) with shaking at 120 rpm, on in King's B agar plates at 28° C. Growth of the anticipated bacteria was noted: orange colonies on KB plates for wild type strain, colorless colonies of the gacS mutant on KB plus kanamycin (25 μg/ml) and orange colonies on KB plus kanamycin and tetracycline (25 μg/ml) of the complemented mutant. Biofilms were grown in the MBEC device following standard methodology.

### Example 2

Scanning Electron Microscopy. After 24 h, pegs were removed from the 96-peg lid of the MBEC device and air dried for 1-2 h at room temperature, under a fume hood. Samples were fixed in 5% glutaraldehyde prepared in 0.1 M sodium cacodylate buffer, pH 7.2, at room temperature. After fixation, pegs were allowed to dry overnight on a Petri-dish, then assembled onto stubs and sputter-coated with gold-palladium. Scanning electron microscopy was performed using a Cambridge Model 360 SEM at 20 kv emission. Digital images were captured from the SEM using OmniVision (v. 5.1) software.

### Example 3

Growth Conditions, Sample Analysis and Bio-Assays of Pseudomonas aeruginosa. Biofilm and planktonic growth studies were performed using the Calgary Biofilm Device (CBD) (MBEC™ Biofilm Technologies Limited). Pseudomonas aeruginosa PA14 wild type, gacA and toxA strains were grown for 24 hours in Tryptic Soy Broth (BDH). Biofilm and planktonic populations were sampled at points.

Sampling of biofilm populations was achieved by dislodging a peg from the 96 peg lid, whereas planktonic populations were sampled by removing an aliquot from the growth vessel. Biofilms were disrupted to release individual component cells by sonication. Cell counts of both populations were determined by serial dilution in 0.9% saline and spot plating on Tryptic Soy Agar plates (BDH). Antibiotic susceptibility profiling of P. aeruginosa PA14 wild type, toxA and gacA strains was performed using the MBEC™ device as per manufacturer's instructions (MBEC™ Biofilm Technologies Limited).

To assess for alterations in the levels of autoinducer production, bio-assays were performed on P. aeruginosa PA14 wild type, PA14 toxA, and PA14 gacA using the reporter strain E. coli MG4 (pKDT17).

To assess for alerations in type IV pili mediated twitching motility of P. aeruginosa PA14 gacA compared to wild type PA14 or the control knock-out strain PA14-toxA, zones of twitching were measured and compared. On very thin LB or TSA plates (<2 mm thick), each of the three PA14 derivative strains were inoculated using a stab loop. Bacterial proliferation between the agar and the plate was measured as the zone of twitching.

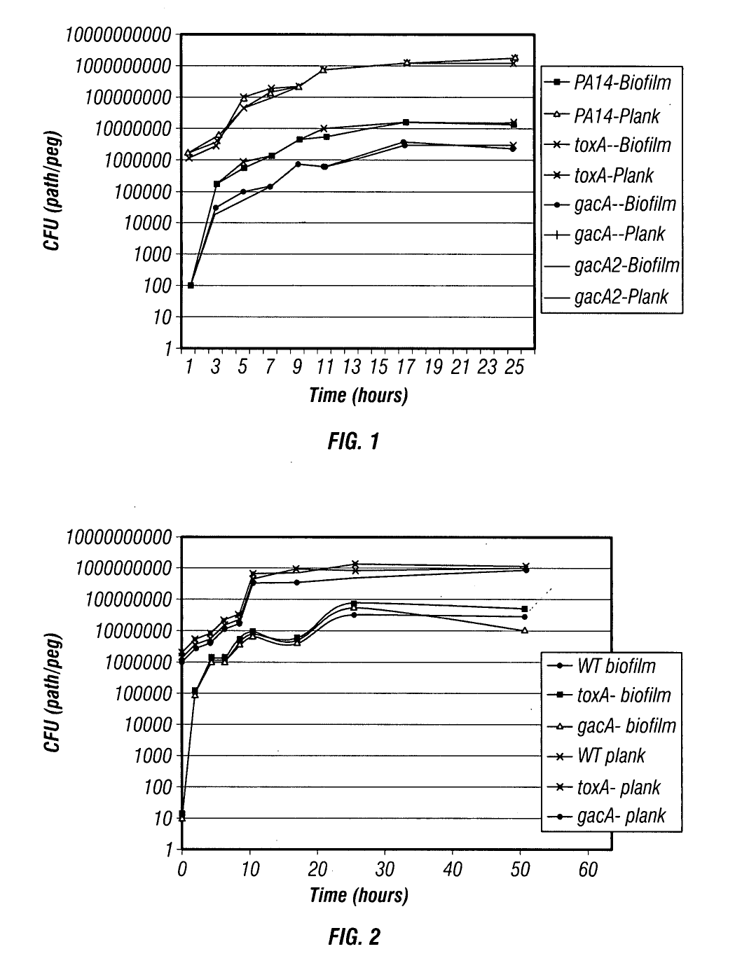

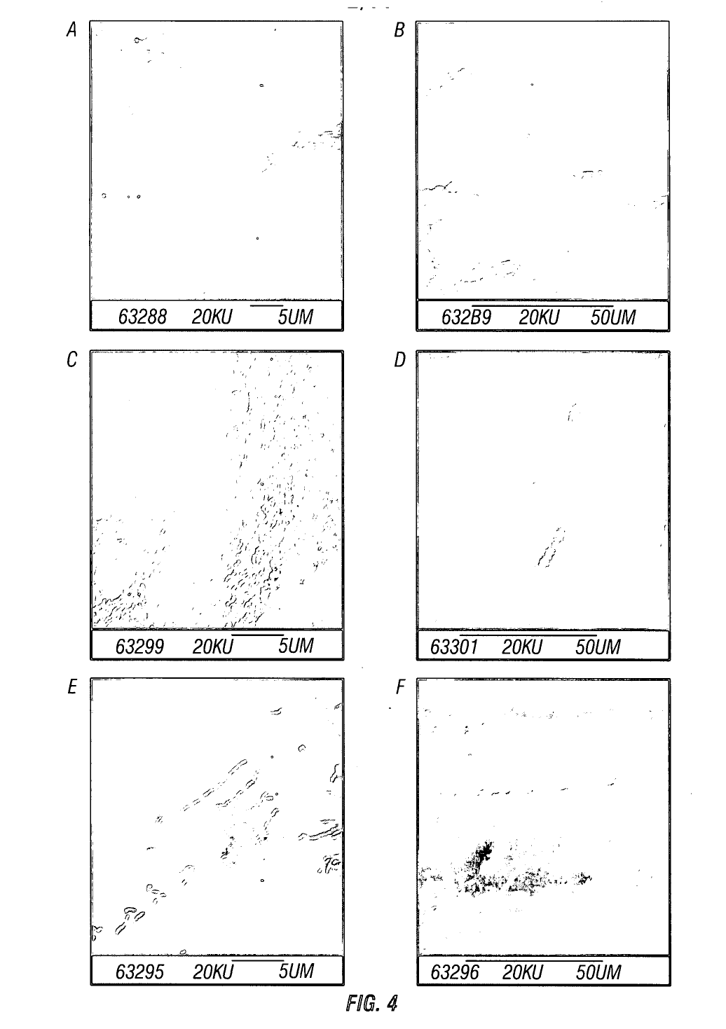

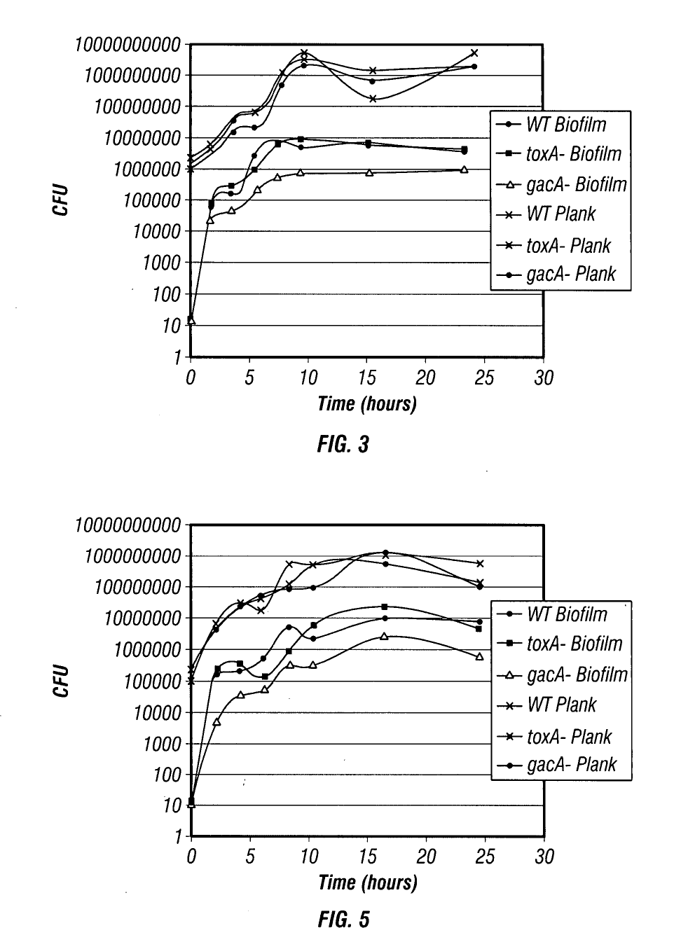

Biofilm growth curves demonstrated that when the response regulator of the two component regulatory system, gacA, was disrupted in P. aeruginosa strain PA14, a 10-fold reduction in biofilm formation ensued relative to wild type PA14 and a toxA derivative. This reduction in biofilm formation was evident in both the rate at which biofilms were formed over a 24 hour time period as well as final biofilm size. However, no significant difference in the planktonic growth rate of PA14 gacA was observed compared to the two control strains (See FIG. 1). When gacA was provided in trans in the multi-copy vector pGacA to strain PA14 gacA, the defect in biofilm formation ability was abrogated (See FIG. 2). The biofilm formation defect was not corrected in PA14 gacA when transformed with the control vector pUCSF (See FIG. 3).

Scanning electron microscopy of biofilms formed by PA14 gacA revealed diffuse clusters of adherent cells which failed to aggregate into microcolonies. Biofilms formed by wild type PA14 or the control toxA derivative had normal biofilm characteristics and formed a dense mat of bacterial growth. This evidence implies that the gacA knock-out strain of PA14 has an inherent defect in biofilm maturation, the result of disrupting the GacA/GacS regulon (See FIG. 4).

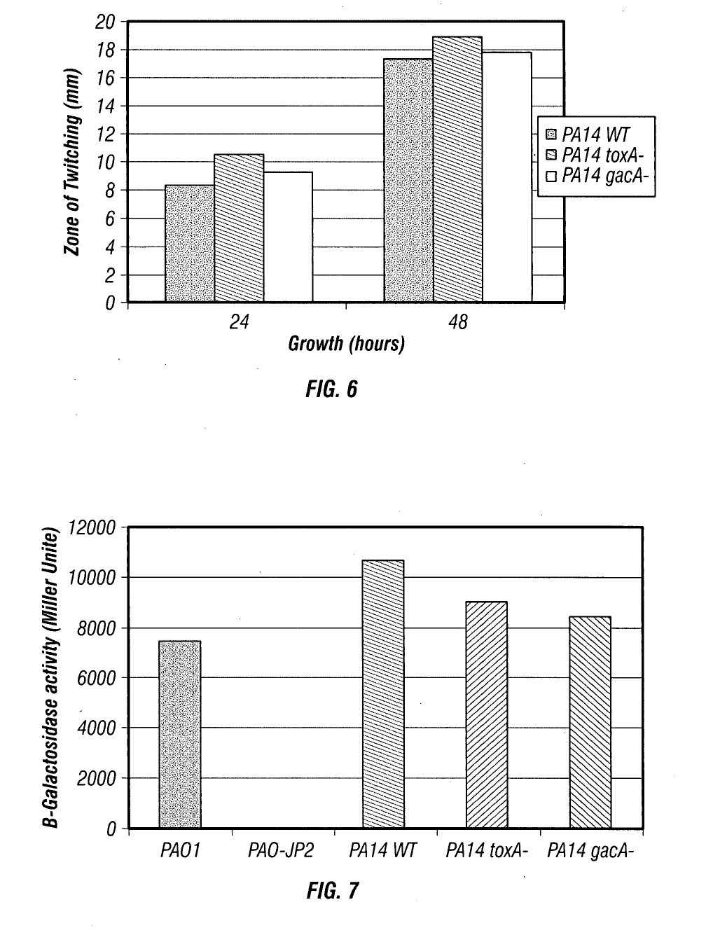

To ensure that the defect in biofilm formation ability caused by the disruption of the GacA/GacS regulon of P. aeruginosa is not merely an upstream effect acting on factors already identified to be involved in biofilm formation, several bioassays were performed. Growth curves were performed on strains PA14, PA14 toxA and gacA transformed with pMJG1.7, a multi-copy vector expressing lasR. Over-expression of lasR did not complement the biofilm formation defect of strain PA14 gacA (See FIG. 5). LasR is the transcriptional activator of the las quorum sensing system demonstrated to be necessary for biofilm maturation. Twitching motility assays revealed that P. aeruginosa PA14 gacA does not have altered twitching motility mediated by type IV pili relative to either control strains (See FIG. 6). Twitching motility has been shown to be necessary for cellular aggregation to form microcolonies, during the initial steps of biofilm formation. Bioassays used to detect the level of autoinducer production in P. aeruginosa demonstrated that PA14 gacA does not have significantly altered levels of N-3-oxododecanoyl-L-homoserine lactone (PAI-1; C12 homoserine lactone (HSL)) relative to the two control strains. C12 homoserine lactone (HSL) has been shown to be required for microcolony maturation into fully developed biofilms (See FIG. 7). The results of these studies confirm that the gacA/gacS regulon itself, and not downstream factors previously identified in biofilm formation, is responsible for the biofilm formation defect of P. aeruginosa PA14 gacA.

Antibiotic susceptibility profiling has demonstrated PA14 gacA biofilms have moderately decreased resistance to azythromycin, chloramphenicol, erythromycin, piperacillin, and polymixin B relative to either PA14 wild type or the toxA control strain.

These findings clearly demonstrate a role for the GacA/GacS two component regulatory system of P. aeruginosa in biofilm formation. Disruption of biofilm formation by targeting the GacA/GacS two component regulatory system is a therapeutic treatment for cystic fibrosis pulmonary infections.

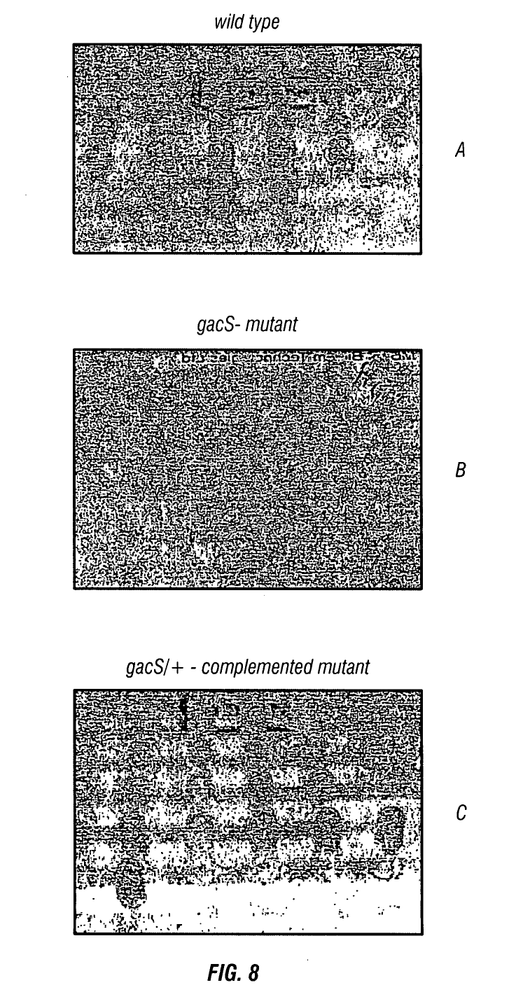

As shown at FIG. 8, when the response regulator of the two component regulatory system, gacS, was disrupted in a gacS knock-out mutant of P. chlororaphis O6, a complete suppression of biofilm formation on MBEC device ensued relative to wild type PcO6. When gacS was provided in trans in the multi-copy vector pGacS to strain PcO6gacS, the defect in biofilm formation ability was abrogated (See FIG. 8).

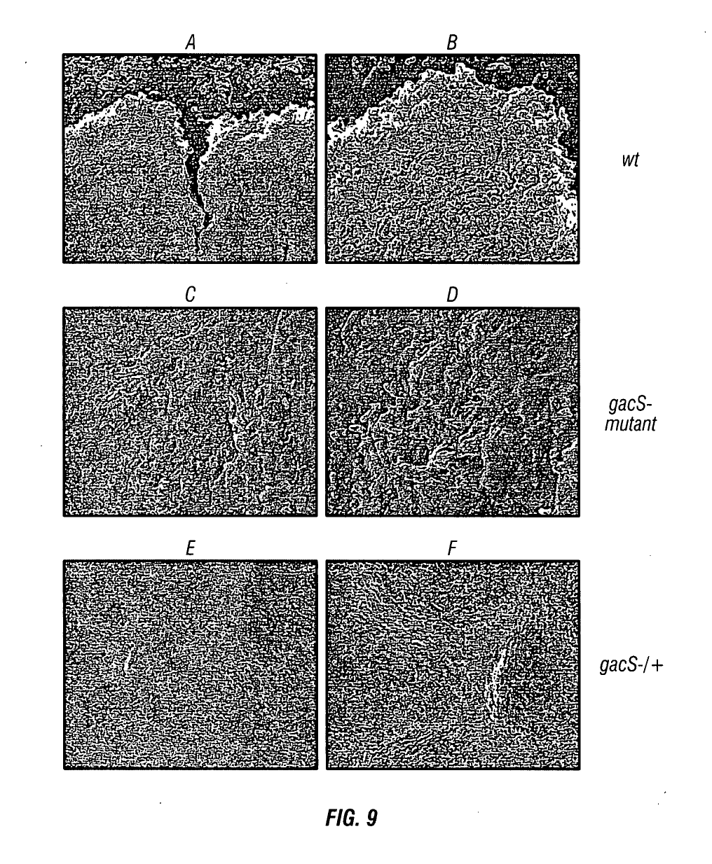

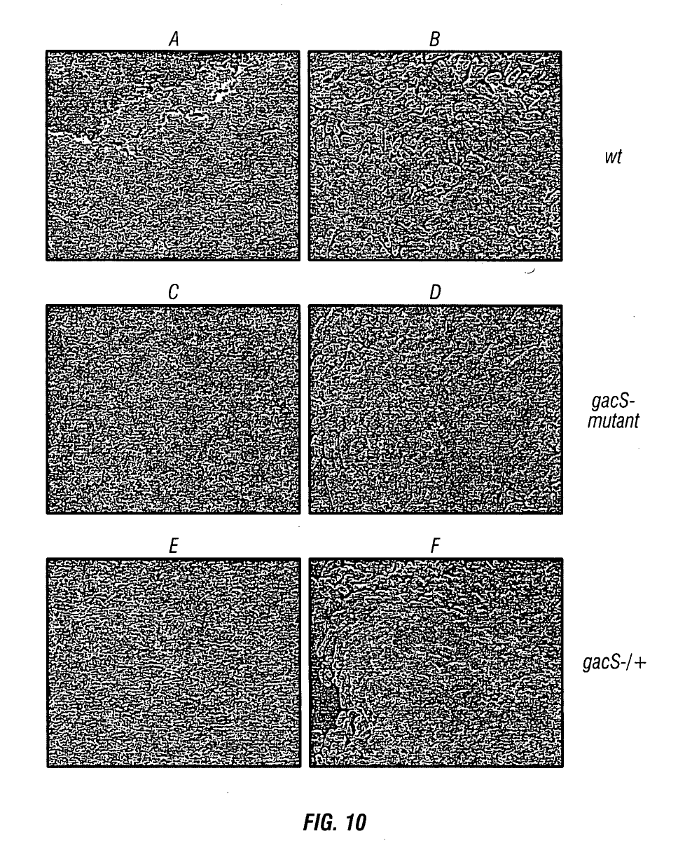

Scanning electron microscopy of biofilms formed by PcO6gacS revealed diffuse clusters of adherent cells which failed to aggregate into microcolonies. (FIGS. 9 and 10C, D). Biofilms formed by wild type PcO6 (FIGS. 9 and 10A, B) or gacS/+−complemented mutant (FIGS. 9 and 10E, F) had normal biofilm characteristics and formed a dense mat of bacterial growth. This evidence implies that a gacS knock-out mutant of P. chlororaphis O6 has an inherent defect in biofilm maturation, the result of disrupting the GacA/GacS regulon.

In natural environments or within a host, bacteria associate with surfaces to form polymer-enclosed biofilm. Pseudomonas aeruginosa is successful at adapting to thwart biological or chemical removal. In a wide variety of environmental niches such as soil, water, plants later stages of development, community growth and behaviour is coordinated by quorum sensing, a process that relies on intercellular signaling by N-acylhomoserine lactones (AHSLs). In P. aeruginosa, GacA is a positive transcriptional regulator of the lasRI and rhlRI operons, which are responsible for the enzymes that synthesise N-3-oxo-dodecanoyl-homoserine lactone (3-oxo-C12-HSL) and N-butanoyl-homoserine lactone (C4-HSL), respectively. Loss-of-function mutations in gacS and/or gacA in Pseudomonas species reduce production of these auto-inducers. In vitro, these quorum-sensing systems are pivotal for P. aeruginosa biofilm tolerance to hydrogen peroxide, amino-glycoside antibiotic, and polymorphonuclear leukocytes. It s a paradox that, despite a role in stress tolerance and survival, spontaneous mutations in gacS and/or gacA have been observed in many pseudomonads under laboratory conditions as well as in the plant rhizosphere.

Bacterial strains used in this study are summarized in Table 1. All strains were stored at 70° C. in Microbank™ vials (Pro-Lab Diagnostics, Toronto, Canada) according to the manufacturer's instructions. Unless otherwise noted, P. aeruginosa and Escherichia coli were grown in tryptic soy broth or Miller Luria-Bertani broth (TSB and LB, respectively, Difco, Franklin Lakes) at 35° C. Alternatively, nutrient agar or Miller Luria-Bertani agar (Difco) was used to culture these bacteria. Antibiotics and sucrose were added as selective agents where appropriate. Viable cell counting was performed by 10-fold serial dilution of cultures in phosphate-buffered saline (pH 7.2) and subsequent plating onto agar medium.

TABLE 1

Bacterial strains, plasmids and PCR primers used in this study

| | | |
|---|---|---|
| Strain or plasmid | Genotype or description | Source |

| | | |
|---|---|---|
| Escherichia | | |
| coli | | |
| JM109 | endA1•recA1•gyrA96•hsdR17(r κ̅m κ̅)•supE44•recA1A(lac-proAB); | Yanisch-Perron et al. |
| | F′(traD36•proAB⁺•lacF′•lacZ•M15) | (1985) |
| XL1 Blue | endA1•recA1•gyrA96•thi1•hsdR17•relA1•supE44•lac; | Bullock et al. (1987) |
| | F′[proAB, lacF′ZΔM15 Tn10(Tet′)] | |
| DH5α | supE44•hsdR17•Δ(lac)U169•recA1-endA1•gyrA96•thi1•relA1•deoR | Hanahan (1983) |
| | (φ80lacZΔM15) | |
| SM10 | thi1•recA1•thr•leu•tonA•lacY•supE44•RP4-2-Tc::Mu::pir | Simon et al. (1983) |
| MG4 | Reporter Strain | Pearson et al. (1994) |
| Pseudomonas | | |
| aeruginosa | | |
| PAO-JP2 | Δrhll::Tn501 derivative of wild-type PAO1, Δlasl, Hg^{R} Tc^{R} | Pearson et al. (1995) |
| UCB-PP PA14 | Clinical isolate | Rahme et al. (1995) |
| PA14 gacA⁻ | PA14ΔgacA::gm^{r} | Rahme et al. (1995) |
| PA14 gacS⁻ | PA14ΔgacS::gm^{r} | This study |
| PA14 SCV | PA14ΔgacS::gm^{r} small colony variant | This study |
| Plasmids | | |
| pBluescriptll ks+ | Cloning and sequencing vector, amp^{R} | Stratagene |
| pBSllgacS | pBluescriptll ks + containing a 2.0 kb portion | This study |
| | of gacS amplified from PA14 genome; amp^{R} | |
| pBSllgacS::gm | pBSllgacS containing the gm^{R} cassette from pUCGM | This study |
| | in the gacS region; gm^{R} | |
| pEX18 | Used for allelic exchange mutagenesis. | |
| | Constructed by ligation of 1791 bp Avul fragment | Hoang et al. (1998) |
| | of pUC18 to large Pvul fragment of pEX100T; amp^{R} | |
| pEX18gacS::gm | pEX18 containing the gacS::gm region from | |
| | pBSllgacS::gm; amp^{R}, gm^{R} | This study |
| pUCGM | Plasmid containing Tn1696 derived gm^{R} gene | Schweizer (1993) |
| | flanked by pUC19 polylinker site; amp^{R} gm^{R} | |
| pUCP18 | 1.8-kb stabilizing fragment from P. aeruginosa | Schweizer (1991) |
| | incorporated into pUC18 | |
| pUCP18mpgacS | pUCP18 containing a 3.4-kb fragment amplified | This study |
| | from P. aeruginosa PA14 containing the | |
| | entire gacS gene and flanking sequences | |
| pECP61.5 | rhlA::lacZ reporter construct | Pearson et al. (1995) |
| pKDT17 | lasB::lacZ reporter construct | Pearson et al. (1994) |
| PCR primers | | |
| Prod7 Forward | 5′-GATGGTGCTTGGCGGTTACTTCAC-3′ | This study |
| Prod7 Reverse | 5′-ACGTCCATGAAGACCAGGTCGAAG-3′ | This study |
| MPGACS Forward | 5′-CGCCAACCCCTCTTCCCCGTCTC-3′ | This Study |
| MPGACS Reverse | 5′-CGGCGACAGCGTGCGGCGAATAG-3′ | This study |

Plasmid constructs and strain generation. The plasmids and PCR primers used in this study are summarized in Table 1. A 2-kb fragment of the gacS gene was amplified by PCR (94° C. for 5 min, then 35 cycles consisting of 94° C. for 30 s, 65° C. for 30 s, 72° C. for 2 min, followed by a terminal incubation at 72° C. for 7 min, then held at 15° C.) from P. aeruginosa PA14 genomic DNA using Platinum Pfx polymerase (Invitrogen, Carlsbad, Calif.) and Prod7 forward and reverse primers. Once amplified, the fragment was isolated and ligated into the EcoRV site of pBluescript II ks1(Stratagene, La Jolla, Calif.) to produce the interim plasmid construct pBSIIgacS.

The gentamicin resistance (gmr) cassette from pUCGM (Schweizer, 1993) was inserted into the SphI site of the gacS fragment of pBSIIgacS. A 3-kb fragment comprising the original gacS fragment and the gmr cassette was then amplified by PCR and incorporated into the SmaI site of pEX18 (Hoang et al., 1998) to produce plasmid pEX18gacS::gm, which was transformed into E. coli SM10. The plasmid was then transferred by conjugation to P. aeruginosa PA14. Overnight cultures of P. aeruginosa PA14 and E. coli SM10 (pEX18gacS::gm) were grown in LB broth (Sambrook & Russell, 1989) containing no antibiotics and 15 mg/mL gentamicin, respectively. Cells were pelletted by centrifugation (800 g for 5 min), gently resuspended in a small volume of phosphate-buffered saline (PBS) and combined so as to have donor cells in excess of recipients. The cell mixture was spotted onto TY plates (8 g tryptone, 5 g select yeast extract, 2.5 g NaCl/L agar) and incubated overnight at 37° C. Isolation of a gacS mutant was accomplished through selection for spontaneous allelic exchange events that transferred the gmr cassette from pEX18gacS::gm into the genomic gacS gene. The resulting lawn of cells was scraped from the TY plate, resuspended in PBS, and deposited onto Vogel Bonner minimal media plates containing 15 pg/mL gentamicin. Potential mutants were then assessed for sucrose sensitivity (5% sucrose in LB agar) to confirm loss of the donor plasmid (pEX18) (Yanisch-Perron et al., 1985).

The plasmid pUCP18mpgacS was constructed in order to complement P. aeruginosa gacS mutants with exogenous gacS in trans. The entire gacS gene, plus c. 300 bp of flanking DNA, was amplified by PCR using Platinum Pfx polymerase (Invitrogen) and the MPGACS forward and reverse primers. This fragment was ligated into the SmaI site of pUCP18 and introduced into the P. aeruginosa gacS mutant via conjugation with transformed E. coli SM10. Pseudomonas aeruginosa clones carrying the pUCP18mpgacS construct were identified by carbenicillin resistance (500 μg/mL), plasmid isolation, and the amplification of appropriately sized PCR products.

Biofilm formation. Biofilms were aerobically cultivated using the MBEC high-throughput (HTP) or Physiology and Genetics (P&G) device (Innovotech, Edmonton, Canada) as described in the manufacturer's instructions and by Ceri et al. (1999). To summarize, the parts of this batch culture apparatus were used in two ways. The top half of the plastic MBEC™-HTP device is a lid with 96 polystyrene pegs that also fits over a standard 96-well microtitre plate. For Biofilm growth, the bottom half was either (1) a corrugated trough that guided 22 mL of inoculum across the pegs when the device was placed on a rocker at 3.5 rocks per min (HTP format), or (2) a microtitre plate with 150 mL of inoculum in each well that was placed on a gyrorotary shaker at 150 r.p.m. (P&G format). For either assay format, the inoculum was prepared to c. 10⁷CFU/mL1 of the desired strain and the inoculated device was incubated at 35° C. and 95% relative humidity for the required time. Unless otherwise noted, all experiments utilized the MBEC P&G assay. Pegs from the devices were collected at specific time points for scanning electron microscopy and scanning confocal laser microscopy. Biofilm cell densities were evaluated by breaking pegs from the lid of the MBEC device with sterile pliers, rinsing the peg in PBS, and subsequent viable-cell counting as described above. PBS containing the anionic surfactant Tween-20 (1% v/v) was used to assist in recovery of bacteria from the peg surfaces. Pegs were sonicated for 30 min in an Aquasonic model 250HT ultrasonic cleaner (VWR International, Mississauga, Canada). Samples of broth culture were collected at the same time points and viable cell counts were determined in a similar manner.

Swim and swarm assays. Swim assays were performed on a semisolid medium composed of Miller LB broth amended with 0.3% agar per litre. Swarm assays were carried out on a modified M9 medium, containing per litre of double-distilled water 3.0 g KH2PO4, 6.0 g Na₂HPO₄, 0.5 g NaCl, 0.5 g L-glutamate, 2.0 g dextrose, and 5.0 g agar. This medium was autoclaved and enriched with 1 mL of 1 M MgSO₄ and 1 mL of 0.01 M CaCl₂. One microlitre aliquots of overnight bacterial cultures were spotted into the middle of the swim or swarm plates, which were incubated for 72 h at room temperature and 35° C., respectively. Swim diameter was measured and plates were photographed using a Kodak EasyShare C340 digital camera (Kodak, Toronto, Canada).

Scanning confocal laser microscopy. Three-dimensional (3-D) Biofilm structure was evaluated by scanning confocal laser microscopy (SCLM). Pegs were broken from the MBEC device and immersed in 0.1% w/v acridine orange (Sigma Chemical Co., St Louis, Mo.) in PBS for 5 min at room temperature. Acridine orange is a membrane-permeant nucleic acid stain that interchelates dsDNA and binds ssDNA through dye-base stacking. This fluorophore has an excitation wavelength of 488 nm and broad spectrum emission. Biofilms were examined using a Leica DM IRE2 spectral confocal and multiphoton microscope with a Leica TCS SP2 acoustic optical beam splitter (AOBS) (Leica Microsystems, Richmond Hill, Canada). A 63 water immersion objective was used in all imaging experiments. Image capture and 3-D reconstruction of z-stacks were performed using LEICA CONFOCAL SOFTWARE (LCS).

Scanning electron microscopy. Pegs broken from the MBEC device were air-dried for up to 2 h at room temperature, and then fixed for 2 h at 4° C. in a solution of 5% glutaraldehyde in 0.1 M sodium cacodylate buffer (pH 7.2). Samples were air-dried overnight, attached to aluminium stubs using epoxy resin, and then sputter-coated with gold/palladium using a Technics Hummer I sputter coater. Scanning electron microscopy (SEM) was performed using a Cambridge Model 360 SEM at 20 kV emission or an environmental SEM (ESEM) Phillips XL 30 ESEM (Morck et al., 1994). Digital images were captured using OMNIVISION 5.1 software (Omni-Vision Technologies Inc., Sunnyvale). Data shown are representative of over 100 fields of view for each treatment. Treatments and SEM analysis were repeated independently in triplicate, with at least three sampled pegs of each strain viewed at each time period.

N -Acyl-homoserine lactone determination. For quantification of 3-oxo-C₁₂-AHSL and C₄-AHSL, AHSL biosensors E. coli MG4 (pKDT17) (Pearson et al., 1994) and P. aeruginosa PAO-JP2 (pECP61.5) (Pearson et al., 1995) were used. These systems quantify 3-oxo-C₁₂-AHSL and C4-AHSL production based on the measurement of β-galactosidase activity from lasB::lacZ and rhlA::lacZ reporter constructs, respectively.

Stock solutions of antibiotic, metals and neutralizers. Ciprofloxacin was purchased from Bayer (Leverkusen, Germany) and 30% hydrogen peroxide from BDH Inc. With these exceptions, all other metals, antibiotics and neutralizing agents were purchased from Sigma Chemical Co. Stock solutions of metals were prepared in double-distilled water (ddH2O), syringe-filtered, and stored at room temperature. With the exception of erythromycin, antibiotics were also prepared in ddH₂O but were frozen and stored at −70° C. Erythromycin was prepared in 95% ethanol. H₂O₂ was diluted directly from the bottle supplied by the manufacturer. Challenge media (containing the desired antibacterial) were made up in LB 30 min prior to use. Reduced glutathione (GSH) and L-cysteine were prepared at 0.25 M in ddH₂O, syringe-filtered and stored at 20° C. These two compounds were used at a final concentration of 5 mM each in recovery media for all assays requiring viable cell counting.

Antimicrobial susceptibility testing. Antibiotic, metal and biocide susceptibility tests were performed. Antimicrobials were arranged into arrays in microtitre plates that typically consisted of serial two-fold dilutions along the rows of wells (the challenge plates). The first and last wells of every row were used as sterility and growth controls, respectively. The cultivation times for biofilms used in these assays were calibrated using growth-curve data so that the different strains of P. aeruginosa PA14 produced biofilms with similar viable cell counts. Biofilms were rinsed with PBS (to remove loosely adherent planktonic cells) and inserted into the challenge plates. After exposure, biofilms were rinsed once in PBS and inserted into microtitre plates containing 200 mL of recovery medium in each well (LB broth, 5 mM GSH, 5 mM L-cys, 1% v/v tween-20). These biofilms were disrupted into the recovery medium using a sonicator, and the recovered cells were serially diluted and plated for viable cell counting. Spot plates from these experiments were incubated for a minimum of 36 h at 35° C. before enumeration. Minimum inhibitory concentration (MIC) values were determined by reading the optical density at 650 nm (OD₆₅₀) of challenge plates after 72 h at 35° C. using a THERMOmax microplate reader with SOFTMAX PRO data analysis software (Molecular Devices, Sunnyvale, Calif.).

In an alternative set of experiments, the frequency of SCV cells arising from 24-h biofilms of P. aeruginosa PA14 gacS was evaluated. Antibiotics were diluted from stock solutions into LB broth to obtain final concentrations of 5 or 1.25 pg/mL. These were arranged in triplicate in a micro-titre plate. Biofilms were incubated in these low concentrations of antibacterials for 18 h at 35° C. and 95% relative humidity. Goat and human serum were also assayed in this array, and were kind gifts from The Life and Environmental Sciences Animal Care Facility at the University of Calgary, Department of Biological Sciences. After exposure, biofilms were treated in a manner identical to that described above.

Statistical analysis. One-way analysis of variance (ANOVA) and two-sample t-tests were performed using MINITAB s Release 14 (Minitab Inc., State College, Pa.). Alternative hypotheses were tested at the 95% level of confidence. Mean and standard deviation calculations were performed using Microsoft's Excel 2003 (Microsoft Corporation, Redmond).

Creation of a gacS cell line from P. aeruginosa PA14. The gacS gene was inactivated by allelic exchange for a gentamicin resistance marker from a donor plasmid containing sacB. PA14 cells that were sucrose-resistant (which selected for loss of the donor plasmid) and gentamicin-resistant were assessed for interruption of gacS by determining the size and sequence of the PCR product based on primers corresponding to the gacS gene. The resulting cell line was thus verified by the production of appropriately sized PCR products, and this engineered strain was denoted P. aeruginosa PA14 gacS.

When disrupted and plated onto agar, solid-surface-attached biofilms of P. aeruginosa PA14 gacS that were older than 24 h produced two distinct colony morphologies. After overnight incubation at 35° C., the majority of these colonies were shiny, smooth, light yellow or pale green, and 3-5 mm in diameter. These colonies were similar to those produced by the wild-type organism, with the exception of the slightly greater colony diameter produced by the mutant. A minority of colonies exhibited abrupt edges and were much smaller than colonies produced by either wild-type or gacS- strains of P. aeruginosa PA14. These colonies represented a SCV of the original gacS⁻ strain.

These SCV isolates were evaluated for growth on Pseudomonas isolation agar and for gentamicin resistance (the marker for the gacS mutation), as well as by PCR analysis and Gram-staining. These tests were consistent with the premise that the variants were derived from the parental gacS⁻ strain. The SCVs were stable and no reversion to normal colony morphology was observed, even after three days' incubation in broth medium or 45 days' serial culture on nutrient agar at room temperature. Phenotypically stable SCVs were not observed originating from cultures of wild-type P. aeruginosa PA14 or the isogenic PA14 gacA mutant. Rather, these strains produced colony variants that reverted to the normal colony morphotype after subculture on LB agar (this was replicated five to 20 times for each strain). Further, when PA14 SCV was transformed with the plasmid pUCP18mpgacS (bearing the wild-type gacS gene and flanking DNA sequences), the SCV reverted to the wild-type colony morphology with a frequency of c.10⁻¹.

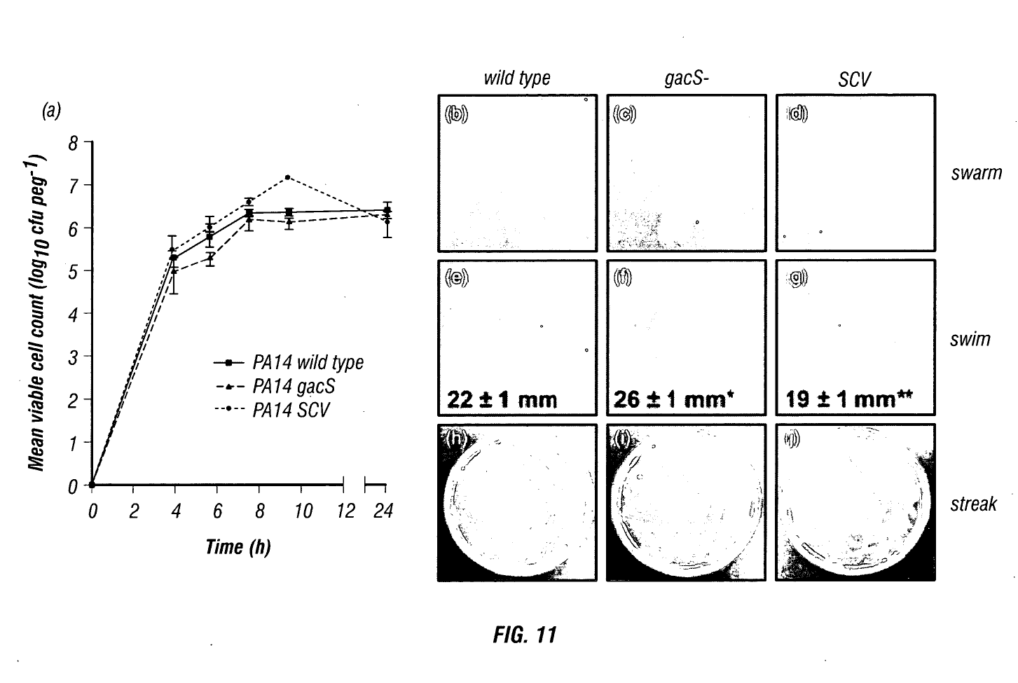

Strain characterization and biofilm formation. Inactivation of the response regulator GacA affects the ability of P. aeruginosa PA14 to form biofilms. Thus, a first logical step was to evaluate Biofilm development by P. aeruginosa PA14 wild-type (PA14 wt), the gacS sensor kinase mutant (PA14 gacS), and the isolated SCV (PA14 SCV). Relative to either PA14 wt or PA14 gacS, PA14 SCV produced biofilms of greater cell density between 4 and 10 h of growth in LB medium (FIG. 11a). By 24 h, these three strains produced biofilms with an equivalent mean viable cell count. However, the biomasses produced by these three strains were not equal. For instance, the extracellular polymeric substance produced by the SCV strain was visible to the naked eye. This difference in biomass was also observed by microscopy. There were no strain differences in the rates of planktonic cell growth. In broth culture, PA14 SCV had a qualitatively greater tendency to form aggregates as well as a surface pellicle.

Each strain of P. aeruginosa was tested for a potential to swarm (FIG. 11b-d) or swim (FIG. 11e-g). PA14 gacS was highly motile relative to the other two strains, and showed an increased ability to swarm. This strain also had significantly greater motility on semisolid swim agar than either PA14 wt or SCV strains (P <0.01, by a two-sample t-test, based on four replicates each). Conversely, PA14 SCV showed significantly decreased swim motility relative to the wild-type strain (P <0.02, by a two-sample t-test, based on four replicates each). In summary, these results show that PA14 gacS is hypermotile and a poor Biofilm former, whereas the isogenic SCV strain is less motile but an excellent Biofilm former. Streak plates of the PA14 wt, gacS⁻ and SCV strains are also pictured in FIG. 11h-j, respectively.

Biofilm structure. Biofilms were examined in situ on pegs from the MBEC device using scanning confocal laser microscopy (SCLM). All bacteria were stained with acridine orange, a membrane permeant nucleic acid interchelator that has broad spectrum fluorescence. This compound stains all cells in a biofilm, live or dead, and may also bind to nucleic acids that are present in the extracellular matrix. Thus, acridine orange may function as a general indicator of biomass present on pegs. Here, surface-adherent growth from P. aeruginosa PA14 wt, gacS- and SCV strains was evaluated after 10 and 24 h. Every image presented here is a representative of at least three independent replicates.

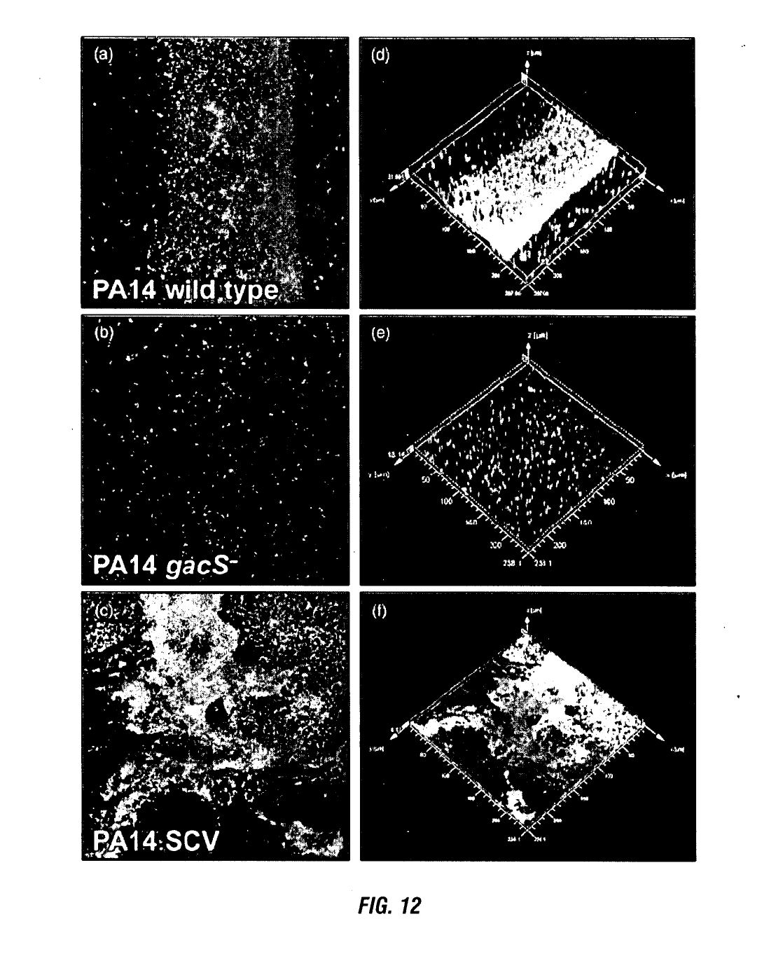

By 10 h, wild-type P. aeruginosa PA14 had formed thin layers of bacteria that were 5-7 mm in height at the air-liquid-surface interface of the polystyrene peg (FIG. 12a and d). In contrast, the gacS- strain had adhered to the surface as scattered cells or small cellular aggregates (FIG. 12b and e). Under the same conditions, the PA14 SCV strain had formed biofilms with greater surface coverage than the wild-type strain and developed into flat layers of densely packed cells that were also 5-7 mm in height (FIG. 12c and f).

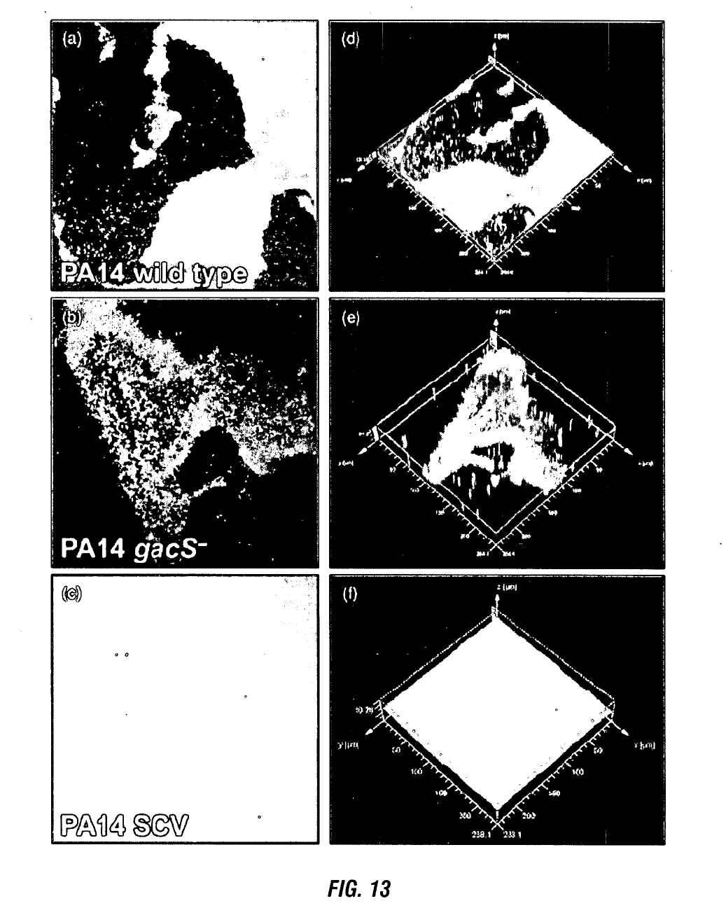

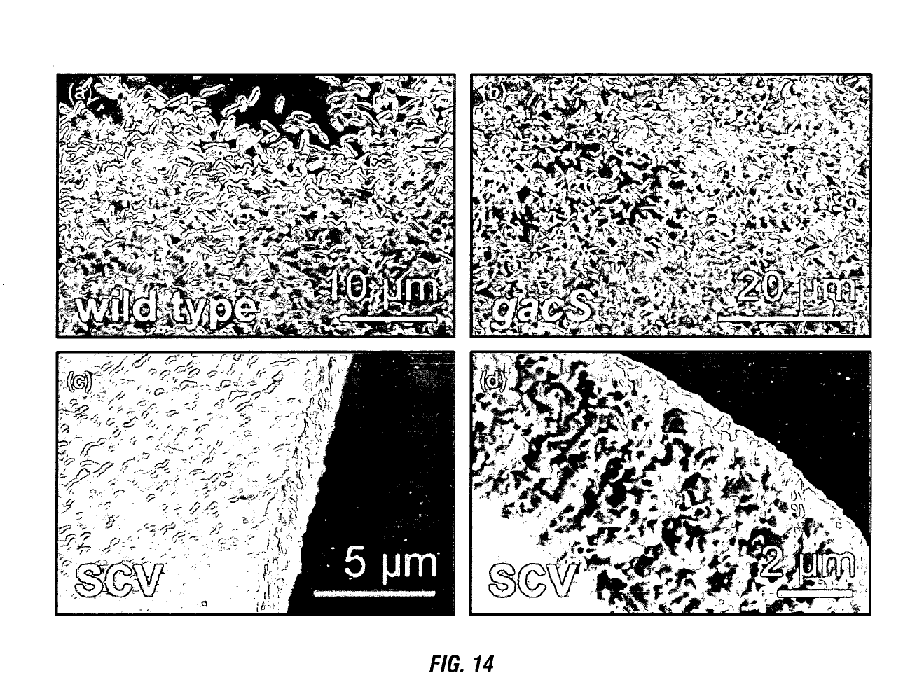

After 24 h of growth, the wild-type strain had formed layers up to 15 mm in height, with the greatest amount of biomass present at the air-liquid-surface interface (FIG. 13a and d). Pseudomonas aeruginosa PA14 gacS formed little more than flat microcolonies and clumps that were heterogeneously distributed across the entire surface (FIG. 13b and e). However, the SCV strain had formed undulating layers of cells that were 20-25 mm thick and that again gave greater surface coverage of the polystyrene pegs than wild-type PA14 (FIG. 13c and f). The structure of biofilms was also examined using SEM at 27 h growth (FIG. 14). These results correlated well with SCLM data at 24 h. In particular, PA14 SCV formed very thick biofilms that lifted away from the surface of the peg when fixed and dehydrated (FIG. 14c and d). At lower magnifications this strain was again observed to produce undulating surface growth owing to the uneven thickness of the Biofilm. As a control, the revertant strain PA14 SCV (pUCP18mpgacS) was similarly imaged using SEM. Biofilms of this revertant covered less surface area and had lost the undulating surface characteristic of the PA14 SCV biofilm. Each SEM image examined here was representative of at least three independent replicates.

N -Acyl-homoserine lactone production. To determine whether there was a correlation between gacS inactivation and AHSL levels, the production of these metabolites was compared between wild-type PA14, gacS⁻ and SCV strains. Pseudomonas aeruginosa PA14 gacA was also assayed, this strain produces lower levels of 3-oxo-C12-AHSL than does the wild-type PA14 strain. Escherichia coli MG4 and P. aeruginosa PAO-JP2, bearing plasmids with either a lasB::lacZ (pKDT17) or rhlA::lacZ (pECP61.5) reporter construct, respectively, were used to quantify 3-oxo-C₁₂-AHSL and C₄-AHSL levels to β-galactosidase activity. These data are summarized in Table 2, and each value presented is the mean and standard deviation of three trials. C₄-AHSL production was similar between P. aeruginosa PA14 wt and its isogenic gacA, gacS- and SCV strains. However, there were noticeable strain differences in 3-oxo-C₁₂-AHSL production. Induction of lasB::lacZ expression by PA14 wt was approximately twofold greater than that of PA14 SCV or PA14 gacA, and at least eight times greater than that of PA14 gacS. In other words, inactivation of gacA produced a different phenotype than did inactivation of gacS. Further, as part of the SCV phenotype, 3-oxo-C₁₂-AHSL production was partially restored (Table 2). These results were corroborated by thin-layer chromatography.

TABLE 2
β-galactosidase reporter activity mediated by N-acylhomoserine
lactones (AHSLs) from overnight cultures of Pseudomonas aeruginosa
PA14 wild-type, mutant and small colony variant (SCV) strains

| | | Pseudomonas aeruginosa PA14 (all values are in Miller units) | | | |
|---|---|---|---|---|---|
| AHSL | Reporter | Wild type | gacS⁻ | gacA⁻ | SCV |
| 3-oxo-C12-HSL | lasB-lacZ | 590 ± 17 | 74 ± 14 | 322 ± 30 | 300 ± 50 |
| C4-HSL | rhlA-lacZ | 297 ± 3 | 250 ± 9 | 245 ± 5 | 313 ± 20 |

Antimicrobial susceptibility. Biofilms are less susceptible to many antimicrobial agents than the corresponding planktonic cells. Mutations in gacA were shown to reduce the resistance of P. aeruginosa PA14 biofilms to some antibiotics. The biofilms of PA14 gacS or PA14 SCV strains were examined to determine whether they had altered resistance to antibacterials relative to the wild-type strain. Here, the inhibitory and bactericidal actions of metal cations (Cu²⁺ and Ag⁺), hydrogen peroxide (H₂O₂) and ciprofloxacin were evaluated. Cu²⁺ and Ag⁺ are industrial pollutants that are also used as disinfectants, H₂O₂ is produced by plant and animal hosts, and ciprofloxacin is an antibiotic clinically used to treat P. aeruginosa infections.

For susceptibility testing, growth-curve data were used to calibrate incubation times to produce biofilms of similar cell density. For these assays, PA14 wt, gacS⁻ and SCV were incubated at 35° C. for 6.0, 7.0, and 5.5 h, respectively, to produce biofilms with cell densities of 5.0±0.7, 5.3±0.5, and 5.5±0.4 log¹⁰ CFU/peg (based on the mean and standard deviation of 50-55 pooled replicates each). Biofilms formed by individual strains in the MBEC P&G device were statistically equivalent between the different rows of pegs (0.09<P<0.91 by one-way analysis of variance). In this model system, planktonic cells shed from the surface of biofilms served as the inoculum for MIC determinations. The advantage of this system is that it may reflect infections or environmental settings where biofilms and planktonic cells form integrated parts of the microbial lifestyle. These data are summarized in Table 3, and each value represents the mean and standard deviation of four independent trials. There were no significant differences in planktonic cell susceptibility to either Cu²⁺, Ag⁺ or ciprofloxacin between the different strains (i.e. there was a log₂ difference or less between these values). However, planktonic PA14 gacS was hypersensitive to H2O2, whereas (by comparison) PA14 SCV was highly resistant.

TABLE 3
Antimicrobial susceptibility of Pseudomonas aeruginosa PA14 wild-type, gacS⁻
and small colony variant (SCV) strains.

| | Wild type | | gacS⁻ | | SCV | |
|---|---|---|---|---|---|---|
| Antibacterial | MIC_{72 h} | MBEC_{99.9} | MIC_{72 h} | MBEC_{99.9} | MIC_{72 h} | MBEC_{99.9} |
| Cu²⁺ (mM) | 8 ± 0 | 8 ± 0 | 8 ± 0 | 7 ± 2 | 4 ± 0 | 64 ± 0 |
| Ag⁺ (mM) | 0.04 ± 0.02 | 0.08 ± 0.05 | 0.04 ± 0.01 | 0.06 ± 0.02 | 0.04 ± 0 | 4.8 ± 0 |
| H₂O₂ (ppm) | 938 ± 0 | 352 ± 135 | 45 ± 16 | 22 ± 14 | 1875 ± 0 | 293 ± 203 |
| Ciprofloxacin (μg mL⁻¹) | 0.4 ± 0.2 | 0.16 ± 0 | 0.7 ± 0.3 | 0.12 ± 0.05 | 0.8 ± 0.4 | 0.64 ± 0.45 |

The anti-Biofilm activity of Cu²⁺, Ag⁺, H₂O₂, and ciprofloxacin was evaluated by determining mean viable cell counts and log-killing of Biofilm populations of P. aeruginosa PA14 wt, gacS⁻ and SCV strains. Consistent with the American Clinical and Laboratory Standards Institute's definitions (CLSI, http:˜www.nccls.org/), the bactericidal threshold was defined as a 3 log₁₀ reduction in viable cells in the bacterial population. This value will be denoted here as the minimum Biofilm eradication concentration required to kill 99.9% of the bacterial cells (MBEC_{99.9}). These values are summarized in Table 3. Although the MBEC_{99.9} values for H₂O₂ are similar for PA14 wt and SCV strains (Table 3), the biofilms of PA14 SCV showed increased survival at subMBEC_{99.9} concentrations relative to the wild-type strain. For ciprofloxacin, Cu²⁺ and Ag⁺, biofilms of the SCV strain were approximately four, eight and 60 times more tolerant to these toxic factors than the wild-type strain.

It was noted that in some instances MIC values obtained using this method were greater than MBEC_{99.9} values. This represents an expected normality, not peculiarity, to the method. For example, over the course of incubation, peroxide would be gradually degraded in the challenge plates, especially by biofilms during exposure. After removing the biofilms from the challenge media, bacteria were allowed to recover for 72 h prior to MIC determination. In contrast, Biofilm cell density was enumerated immediately after exposure to the peroxide (when its in vitro concentration would have been highest). Because there was no corresponding period of recovery for biofilms, this would result in the comparatively lower MBEC_{99.9} value.

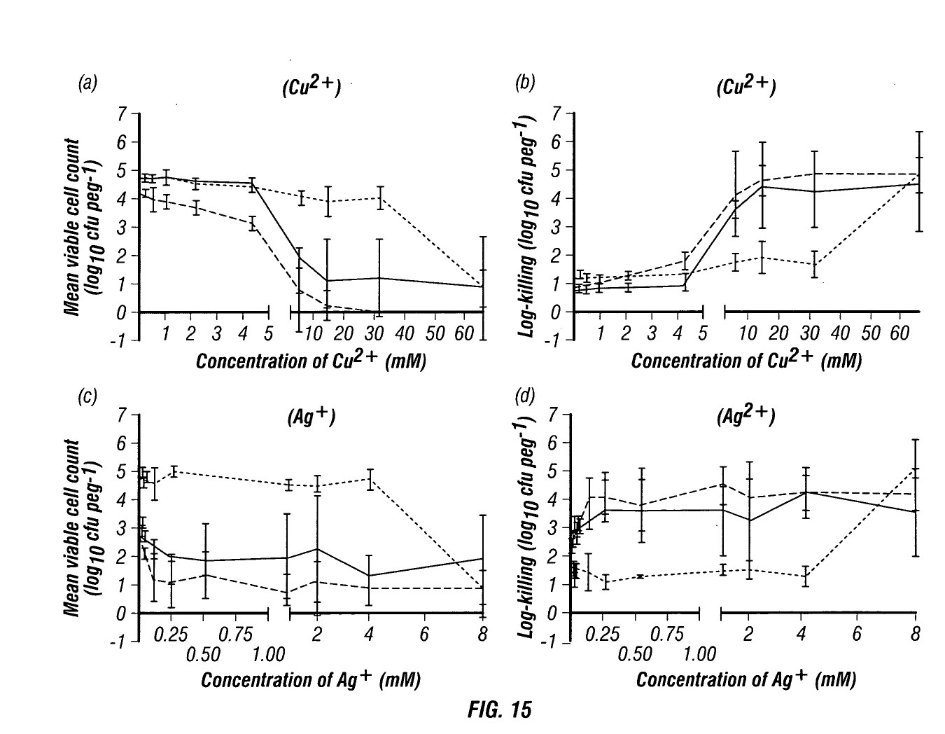

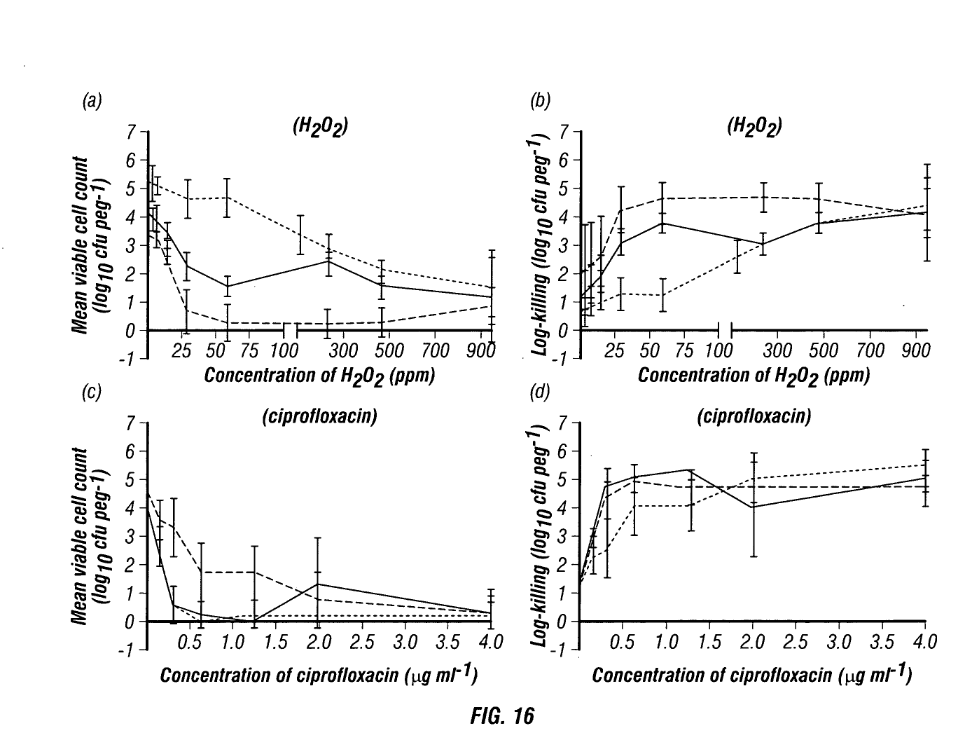

Mean viable cell counts and log-killing data for Cu²⁺ and Ag⁺ are presented in FIG. 15, where each point represents the mean and standard deviation of four independent replicates. Similarly, data for H₂O₂ and ciprofloxacin are presented in FIG. 16. In these two figures, the general trend that P. aeruginosa PA14 gacS is much more susceptible to antimicrobials than the wild-type strain. However, in every case, the PA14 SCV strain produced biofilms that were more tolerant to antimicrobial exposure than those of the wild-type strain. For example, biofilms of the SCV were resistant to 2.4mM Ag⁺, whereas the vast majority of cell viability was lost from PA14 wt and gacS biofilms at 0.04 and 0.02 mM Ag⁺, respectively. Collectively, these data indicate that deletion of gacS reduces the antimicrobial tolerance of P. aeruginosa PA14. However, phenotypic variation in biofilms of this mutant population gives rise to SCV cells that are much more tolerant to antimicrobials than either the wild-type or parental gacS⁻ strain.

Frequency of phenotypic variation. During the course of susceptibility assays, the proportion of SCV cells recovered from biofilms after exposure to Ag⁺ or H₂O₂ was increased relative to the corresponding growth controls. It was queried whether this may be true for other antimicrobial agents or growth conditions. An array of clinically used antibiotics, saline, and goat and human sera were examined for an ability to select for these SCVs from 24-h biofilms of P. aeruginosa PA14 gacS. Biofilms were exposed to these agents for 18 h and each assay was performed in triplicate. Viable cell counts were determined for each exposure condition, and log-survival was determined. The proportion of SCV cells in bacterial populations recovered from these exposure conditions was calculated as the mean of the proportions from each individual trial. The data from these assays are summarized in Table 4.

TABLE 4
Population survival rates and frequency of small colony variants arising from Pseudomonas aeruginosa PA14 gacS⁻
biofilms exposed to antibacterials and various culture conditions

| | | | | | |
|---|---|---|---|---|---|
| | | Concentration | No. of survivors | Log-survival | SCV frequency |
| Test medium | Antibiotic | (μg mL⁻¹) | (log₁₀ CFU peg⁻¹) | (log₁₀ CFU peg⁻¹) | (%) |

Culture conditions (overnight)

| | | | | | |
|---|---|---|---|---|---|
| 0.9% NaCl | None | NA | 3.8 ± 0.3 | −2.5 ± 0.3 | 0 |
| Goat serum | None | NA | 5.4 ± 0.2 | −1.0 ± 0.2 | 4 |
| LB broth | None (growth control) | NA | 6.7 ± 0.2 | +0.3 ± 0.2 | 4 |
| Human serum | None | NA | 5.1 ± 0.2 | −1.3 ± 0.2 | 19 |

Antibiotic exposure (overnight)

| | | | | | |
|---|---|---|---|---|---|
| LB broth | Erythromicin | 5 | 6.4 ± 0.3 | −0.1 ± 0.3 | 0 |
| | Imipenem | 1.25 | 2.7 ± 1.0 | −3.8 ± 1.0 | 0 |
| | Tobramicin | 1.25 | 5.4 ± 0.5 | −1.0 ± 0.5 | 14 |
| | Amikacin | 5 | 3.6 ± 0.6 | −2.8 ± 0.6 | 23 |
| | Azetronam | 1.25 | 4.5 ± 0.5 | −2.6 ± 0.5 | 24 |
| | Ceftrioxone | 1.25 | 6.8 ± 0.5 | +0.4 ± 0.5 | 29 |
| | Oxacilin | 1.25 | 5.6 ± 0.6 | −0.9 ± 0.6 | 32 |
| | Piperacillin + Tazobactam | 5 | 2.7 ± 1.7 | −3.8 ± 1.8 | 33 |
| | Rifampicin | 5 | 5.7 ± 0.3 | −0.7 ± 0.3 | 58 |

Antibacterial exposure (2 h exposure, representative example from susceptibility assays)

| | | | | | |
|---|---|---|---|---|---|
| LB broth | None (growth control) | NA | 4.2 ± 0.3 | −0.9 ± 0.3 | 0 |
| | Ciprofloxacin | 0.16 | 2.4 ± 0.8 | −2.6 ± 0.8 | 0 |
| | Copper cations (Cu²⁻) | 16 | 4.2 ± 0.1 | −0.9 ± 0.1 | 0 |
| | Silver cations (Ag⁺) | 4 | 2.4 ± 0.1 | −2.6 ± 0.1 | 7 |
| | Hydrogen peroxide | 30 | 1.2 ± 1.1 | −3.8 ± 1.1 | 8 |

NA denotes a variable that is not applicable.

Rifampicin, an RNA polymerase inhibitor, was a strong selective agent for SCVs from P. aeruginosa PA14 gacS biofilms. At a concentration of 5 μg/mL, this drug killed 0.7 log₁₀ cells from the Biofilm population. On average, approximately three of five surviving cells from biofilms exposed to this concentration of rifampicin were phenotypic variants. Similarly, the b-lactams piperacillin, oxacillin and ceftrioxone selected for SCVs at a frequency of approximately one in three. This occurred regardless of cell growth (ceftrioxone) or cell death (oxacillin or piperacillin). The aminoglycosides tobramycin and amikacin, both of which find high clinical use in combating P. aeruginosa infections, selected for SCVs at a frequency of approximately one in five. This in vitro selection was compound-specific, as in no instances were saline, erythromycin, imipenem, or ciprofloxacin observed to increase the frequency of SCV cells from PA14 gacS biofilms. Human serum, but not goat, also gave rise to phenotypic variants at elevated frequencies compared with growth controls. These assays indicate that environmental conditions, such as antibacterial exposure or host factors, may select for SCVs from biofilms of P. aeruginosa PA14.

A strain of P. aeruginosa PA14 was created by generating a mutation inactivating the sensor kinase gacS. This mutant was hypermotile and a poor Biofilm former relative to the wild-type strain. While characterizing this strain, it was noted that biofilms of this mutant gave rise to phenotypically stable SCVs at a proportion that was increased by three factors, namely (1) age of the biofilm, (2) by in vitro culture in human serum, and (3) by exposure of biofilms to certain antibacterial agents. This SCV strain had a hyperbiofilm-forming phenotype, and was less motile and more tolerant to bactericidal agents than the parental gacS and wild-type strains. With the exception of phenotypic stability, all of these traits have been described for P. aeruginosa colony morphology variants in the literature. Although there may be multiple mechanisms that give rise to SCVs in P. aeruginosa, thus GacS regulates the reversion of variants to normal colony morphotypes for at least one of these pathways. This premise was supported by complementation analysis, in which SCVs reverted to normal colony phenotypes when transformed with a plasmid bearing wild-type gacS. Thus, the inactivation of gacS, which frequently occurs in laboratory and rhizosphere populations of pseudomonads, may lead to the accumulation of stress-resistant SCV cells in P. aeruginosa biofilms.

These findings are important with respect to the phenotypic variation of P. aeruginosa and other Pseudomonas species in soil. For instance, phenotypic variation in P. fluorescens is mediated by two site-specific recombinases, XerD and Sss, which appear to introduce mutations into gacA and/or gacS. Over-expression of xerD and sss has been used to generate highly motile variants that have an enhanced ability to colonize the alfalfa rhizosphere. Pseudomonas aeruginosa PA14 similarly possesses a homologue of sss (Pseudomonas Genome Database version 2, Locus ID PA14_{—}69710, http:˜v2.pseudomonas.com/) and xerD (PA14_{—}16040). It is worth noting that other rhizosphere Pseudomonas species show phenotypic variation that is based on spontaneous mutation of the gacA and gacS genes that may enhance plant-root colonization. The disclosure provides a link between an inactivating mutation in gacS to the production of stable SCVs in P. aeruginosa. Similarly, the production of these stable colony variants was observed in a DgacS⁻ strain of P. chlororaphis, which characteristically occurs by exposing biofilms to Ag⁺. SCVs of P. chlororaphis O6 generated in this manner show enhanced resistance to certain heavy metals. In conjunction with the data presented in this paper, this affirms the notion that the SCV phenotype may play a role in stress tolerance.

Genes of the GacS regulon strongly influence the later stages of Biofilm formation in P. aeruginosa PA14. Biofilms formed by the PA14 gacS mutant did not proceed far beyond the irreversible attachment and proliferation stages of development. Biofilms of this mutant remained flat and lacked the characteristic layered structures of the mature biofilms formed by the parental strain. The Biofilm growth process observed here for P. aeruginosa PA14 gacS also differed from that previously reported for PA14 gacA, which failed to form surface-adherent aggregates under similar laboratory conditions.

Quorum-sensing systems may be involved in the process of phenotypic variation, and consequently may be indirectly and partly responsible for alterations in antimicrobial susceptibility. Amongst many other genes, these autoinducers control the expression of superoxide dismutase and catalase, which may account for the hypersensitivity of PA14 gacS to H₂O₂. Compared with the gacS⁻ strain, the AHSL levels were partially restored in the SCV, which coincided with increased tolerance to H₂O₂. The increased production of extracellular polymers associated with the SCV strain may further enhance the protective activity of these enzymes. This may contribute to resistance through a reaction-diffusion phenomenon in which the substrate (H₂O₂) is degraded in the extracellular matrix before penetrating into the depths of the biofilm.

The extra biomass in SCV biofilms may also play a role in Cu²⁺ and Ag⁺ sorption. Sequestration of divalent copper cations in P. aeruginosa biofilms has been evaluated using the organic chelator sodium diethyldithiocarbamate to cause coloured precipitation of the metal. Using this approach, biofilms of the PA14 SCV strain qualitatively observed to adsorb greater Cu²⁺ than either the PA14 wt or gacS⁻ strain. This implies that the production of hyper-biofilm-forming SCVs from a genotypically diverse Pseudomonas population represents a strategy that may give rise to elevated heavy metal resistance at the population level. A similar statement may be made for H₂O₂ and ciprofloxacin.

Because the Biofilm mode of growth is thought to be responsible for persistent infections, these P. aeruginosa SCVs may play an additional role in pathogenesis, in particular the destructive infections of the CF lung. Pseudomonas aeruginosa is also known for causing infections associated with burn wounds and the use of catheters. The disclosure provides that low concentrations of clinically used antibiotics may select for hyper-biofilm-forming SCVs from biofilms of the gacS⁻ strain of this nosocomial pathogen. A similar trend has been previously shown for CF isolates of P. aeruginosa. The disclosure provides that silver ions may be added to this list of triggers and/or selective agents. This is important, as silver compounds are finding renewed use in medicine as antimicrobial surface coatings for bandages and catheters. Thus, an emerging and provocative theme is that antimicrobial chemotherapy may be triggering or selecting for the phenotypic variation in P. aeruginosa biofilms that contributes to drug resistance and the destruction of the chronically infected tissue(s). This type of response would also be advantageous in soil environments, where P. aeruginosa, similar to other pseudomonads, would encounter other antibiotic-producing microorganisms, toxic metals, or H₂O₂ produced by plants.

The stability of many types of biological systems is increased by diversity. For instance, phenotypic diversity arises from genetically identical founding populations of P. fluorescens grown in spatially heterogeneous microcosms. In this instance, the emergence of the hyper-biofilm-forming wrinkly-spreader phenotype allows highly efficient colonization of the air-liquid interface. P. aeruginosa Biofilm communities self-generate genetic diversity through a recA-dependent mechanism. Spontaneous mutations in gacS of P. fluorescens introduced by the site-specific recombinases Sss and XerD are analogous and also contribute to phenotypic variation as well as to fitness. This work suggests that P. aeruginosa Biofilm formation and antibacterial resistance are interrelated with phenotypic variation, which itself may be linked to the underlying genetic diversity of these bacterial populations.

Although the invention has been described with reference to the examples above, it should be understood that various modifications can be made without departing from the spirit of the invention. Accordingly, the invention is limited only by the following claims.

What is claimed is:

1. A method of preventing biofilm formation comprising inhibiting the gacA/gacS regulatory system of an organism.

2. The method of claim 1, wherein the organism is P. aeruginosa.

3. The method of claim 1, wherein the inhibition is produced by antibodies to gacS and/or gacA.

4. The method of claim 1, wherein the inhibition is produced by an inhibitory nucleic acid to gacA and/or gacS.

5. A method of inhibiting the production of small colony variants (SCVs) comprising contacting a bacterial population with an antagonist of the gacA/gacS regulatory system of the bacteria.

6. A composition useful for preventing biofilm formation comprising an antagonist of a gacA/gacS regulatory system in a pharmaceutically acceptable form.

7. The composition of claim 6, wherein the antagonist is an antibody to gacS and/or gacA.

8. The composition of claim 6, wherein the antagonist is an inhibitory nucleic acid of gacA and/or gacS.

9. The composition of claim 6, wherein the compound is a small molecule which inhibits gacS and/or gacA.

10. A method of treating an antimicrobial resistant biofilm, comprising:
contacting a resistant bacteria in the biofilm comprising a mutation in gacS with a gacS agonist, wherein the gacS agonist generates a wild-type phenotype in the resistant bacteria.

11. The method of claim 10, wherein the resistant bacteria comprises a small colony variant.

12. The method of claim 10, wherein the gacS agonist comprises a gacS polypeptide.

13. The method of claim 10, wherein the gacS agonist comprises a gacS polynucleotide expressed in trans in the resistant bacteria.

14. The method of claim 10, wherein the contacting is in vivo.

15. The method of claim 10, wherein the contacting is in vitro.

16. The method of claim 15, wherein the contacting is on a surface suspected of having a resistant bacteria.

17. The method of claim 14, wherein the contacting in vivo is by topical administration.

18. The method of claim 10, wherein the bacteria is gram negative.

19. The method of claim 10, wherein the bacteria is selected from the group consisting of E. coli, P. aeruginosa, and S. typhimurium.

20. The method of claim 10, wherein the agonist is administered in combination with at least one antibiotic.

21. The method of claim 20, wherein the class of antibiotic is selected from the group consisting of aminoglycosides, penicillins, cephalosporins, carbapenems, monobactams, quinolones, tetracyclines, glycopeptides, chloramphenicol, clindamycin, trimethoprim, sulfamethoxazole, nitrofuirantoin, rifampin and mupirocin.

22. The method of claim 21, wherein the antibiotic is selected from the group consisting of amikacin, gentamicin, kanamycin, netilmicin, t-obramycin, streptomycin, azithromycin, clarithromycin, erythromycin, erythromycin estolate/ethylsuccinate/gluceptatellactobionate/stearate, penicillin G, penicillin V, methicillin, nafcillin, oxacillin, cloxacillin, dicloxacillin, ampicillin, amoxicillin, ticarcillin, carbenicillin, mezlocillin, azlocillin, piperacillin, cephalothin, cefazolin, cefaclor, cefamandole, cefoxitin, cefuiroxime, cefonicid, cefmetazole, cefotetan, cefprozil, loracarbef, cefetamet, cefoperazone, cefotaxime, ceftizoxime, ceftriaxone, ceftazidime, cefepime, cefixime, cefpodoxime, cefsulodin, i-mipenem, aztreonam, fleroxacin, nalidixic acid, norfloxacin, ciprofloxacin, ofloxacin, enoxacin, lomefloxacin, cinoxacin, doxycycline, m-inocycline, tetracycline, vancomycin, and teicoplanin.

23. A composition comprising a gacS agonist and a pharmaceutically acceptable carrier.

24. The composition of claim 23, wherein the gacS agonist comprises a gacS polynucleotide.

25. The composition of claim 24, wherein the gacS agonist is in a liposomal formulation.

26. The composition of claim 24, wherein the gacS polynucleotide comprises a plasmid.

27. A method comprising inhibiting biofilm formation, comprising:
contacting bacterial population with a gacS and/or gacA antagonist;
monitoring the bacterial population for the formation of a small colony variant;
contacting the small colony variant with a composition comprising gacS agonist.

28. The method of claim 27, wherein the contacting is in vitro.

29. The method of claim 27, wherein the contacting is in vivo.

30. The method of claim 27, wherein the gacS and/or gacA antagonist comprise a polypeptide, inhibitory nucleic acid or small molecule that inhibits or reduces the production or activity of gacS and/or gacA.

31. The method of claim 27, wherein the gacS agonist comprises a gacS polynucleotide.
